(19) United States
(12) Patent Application Publication
(10) Pub. No.: US 2024/0016837 A1
(43) Pub. Date: Jan. 18, 2024

(54) T-CELL RECEPTOR BINDING TO MR1, AND USE THEREOF

(71) Applicant: EUTILEX CO., LTD., Seoul (KR)

(72) Inventors: Byoung S. Kwon, Gyeonggi-do (KR); Yeong Cheol Kim, Seoul (KR); Kwanghee Kim, Seoul (KR); Sunhee Hwang, Gyeonggi-do (KR); Jiwon Chung, Seoul (KR); Jungyun Lee, Seoul (KR)

(21) Appl. No.: 18/008,941

(22) PCT Filed: Jun. 3, 2021
(86) PCT No.: PCT/IB2021/054848

§ 371 Date: Jul. 27, 2023

Related U.S. Application Data
(60) Provisional application No. 63/118,073, filed on Nov. 25, 2020.
(60) Provisional application No. 63/037,085, filed on Jun. 10, 2020.

(51) Int. Cl. A61K 35/17; C07K 16/28; A61K 39/00

(52) U.S. Cl. CPC A61K 35/17; C07K 16/2833; A61K 39/4632; A61K 39/4611; A61K 39/464411; A61K 2239/13; C07K 2317/565

(57) ABSTRACT

The present invention relates to a novel T-cell receptor binding to MR1, and a use thereof. Unlike a conventional customized anticancer immune T cell therapeutic agent, which are limitedly used depending on cancer type and the expression of cancer antigens according to human leukocyte antigen (HLA) type, T cells in which a T-cell receptor is expressed can be applied to all types of cancer regardless of HLA type.

### TECHNICAL FIELD

The present invention relates to a novel T-cell receptor binding to MHC Class I Related Protein (MR1) and a use thereof, for example, a use for immunotherapy of tumors or cancer. A T-cell expressing the T-cell receptor is applicable to all cancer types regardless of a Human Leukocyte Antigen (HLA) type, unlike existing customized anti-cancer immune T cell therapeutic agents which are limitedly used according to the expression of cancer antigens depending on a cancer type and a HLA type.

### BACKGROUND ART

Currently, in the case of most of anti-cancer immune cell therapeutic agents based on T cells, due to heterogeneity exhibited by an individual HLA type, the use thereof is limited as a customized therapeutic agent only for each patient, and the use of the therapeutic agent is possible only for cancer types expressing specific cancer antigens.

The development and clinical approach of some allogenic anti-cancer immune cell therapeutic agents (allogenic T cells) have been considered, but to this end, genetic manipulation is required and thus, safety as well as efficacy need to be guaranteed. In the case of most of developed allogenic genetically modified T cells, for example, allogenic CAR T cells and TCR-engineered T cells, due to the lack of TCR diversity to attack tumors (particularly, solid cancer), the cells may exhibit limiting anticancer effects or be vulnerable to cancer avoidance or recurrence.

Accordingly, there is a need to develop a T cell therapeutic agent rich in TCR diversity that may be used to treat cancer regardless of the expression of cancer antigens according to a HLA type and a cancer type. Furthermore, manipulation of HLA in these T cells can be used as an allogenic T cell therapeutic agent that can be used for all cancer patients.

Conventional T cells bind to a T cell receptor (TCR) that recognizes a peptide antigen (peptide Ag) presented by a major histocompatibility complex (MHC) molecule. However, recently developed T cells recognize a nonpeptidic antigen (Ag) presented by monomorphic MHC class I-like Ag-presenting molecules, and an MHC class I related protein (MR1) is known as an important MHC class I-like antigen presenting molecule that has the ability of providing the nonpeptidic antigen to T cells (Nature Reviews Immunology volume 20, page 141 (2020)).

The MR1 molecule is a non-polymorphic, non-classical MHC molecule that is highly conserved among most mammalian species. Unlike HLA having diversity in individuals, T cells with a unique TCR that binds to MR1, a single HLA-like molecule expressed on the surface of most cancer cells, do not recognize normal cells, but may selectively attack only cancer cells by binding to the MR1 expressed in the cancer cells.

These findings may provide an opportunity capable of implementing HLA-independent, pan-cancer, and pan-population immunotherapy without diversity in the human population.

Regarding the TCR for MR1, International Publication No. WO 2018/162563 discloses a method for isolating TCR-expressing T cells that bind to MR1 of cancer cells. In addition, International Publication No. WO 2020/053312 discloses a method for preparing TCR-expressing T cells that bind to MR1 of cancer cells. Moreover, International Publication No. WO2019/081902 discloses an MR1 TCR including a specific CDR sequence.

Under this technical background, the present inventors of the present invention confirmed a novel T-cell receptor and a use thereof in which a T cell expressing the T-cell receptor can bind to MR1 which is applicable to all cancer types regardless of a Human Leukocyte Antigen (HLA) type, unlike existing customized anti-cancer immune T cell therapeutic agents, which are used limitedly according to the expression of cancer antigens depending on a cancer type and a HLA type, and then completed the present invention.

### DISCLOSURE

An object of the present invention is to provide a novel T-cell receptor binding to MHC class I related protein (MR1).

Another object of the present invention is to provide a nucleic acid encoding the T-cell receptor.

Yet another object of the present invention is to provide a vector in which the nucleic acid is cloned.

Still another object of the present invention is to provide a T cell expressing the T-cell receptor.

Still yet another object of the present invention is to provide an anti-tumor or anti-cancer composition including the T-cell receptor, the nucleic acid, the vector, or the T cell.

In order to achieve the objects, the present invention provides a T-cell receptor binding to MHC class I related protein (MR1) including at least one CDR3 selected from the group consisting of SEQ ID NOs: 2 to 13.

The present invention provides a nucleic acid encoding the T-cell receptor.

The present invention provides a vector in which the nucleic acid is cloned.

The present invention provides a T cell expressing the T-cell receptor.

The present invention provides an anti-tumor or anti-cancer composition including the T-cell receptor, the nucleic acid, the vector, or the T cell.

### DESCRIPTION OF DRAWINGS

FIG. 1 is a schematic diagram illustrating a specific method for isolating and mass-culturing MR1-restricted cancer-killing CD8+T lymphocytes.

FIG. 2 illustrates a result of confirming that MR1 is expressed at a low level in human melanoma (A375), breast cancer (SKOV-3), colorectal cancer cell lines (SW480, HCT-15), and the like.

FIGS. 3A and 3B illustrate results of isolating and proliferating MR1-restricted T cells based on a proliferation dye.

FIG. 4A illustrates results of confirming that the selected MR1-restricted T cells are not MALT cells.

FIG. 4B illustrates results of confirming 4-1BB expression in MR1-restricted T cells.

FIG. 5 is a schematic diagram illustrating a specific structure of CD8+ T cells.

FIG. 6 is a schematic diagram of a platform technology for producing CD8+ T cells having pan-cancer killing ability.

FIG. 7 illustrates a structure of an MR1 TCR lentiviral plasmid.

FIG. 8 illustrates a structure of a vector backbone for cloning the MR1 TCR lentiviral plasmid.

FIG. 9 illustrates a cloning result of an MR1 TCR lentiviral plasmid transfected plasmid.

FIG. 10 illustrates results of confirming TCR expression in Jurkat-NFAT-Luciferase.

FIG. 11 illustrates results of a functional assay by MR1 activation.

### MODES OF THE INVENTION

Unless defined otherwise, all technical and scientific terms used in the present specification have the same meaning as commonly understood by those skilled in the art to which the present invention pertains. In general, the nomenclature used herein is well-known and commonly used in the art.

According to an aspect, the present invention provides a T-cell receptor binding to MHC class I related protein (MR1) including at least one CDR3 selected from the group consisting of SEQ ID NOs: 2 to 13. The present invention includes specifically, CDR3α selected from the group consisting of SEQ ID NOs: 3, 5, 8, 9, and 13; and CDR3β selected from the group consisting of SEQ ID NOs: 2, 4, 6, 7, 10, 11, and 12.

The term T-cell receptor (TCR) in the present invention relates to a TCR or functional fragment and its polypeptide, which includes a chain consisting of a unique combination of domains designated with variable (V), diversity (D), junction (J), and constant (C). The T-cell receptor may include cellular functional fragments of TCR α and β chains, for example, those linked by disulfide bonds but lacking transmembrane and cytosolic domains.

In a T-cell clone, the combination of the V, D, and J domains of α and β chains or δ and γ chains participates in antigen recognition in a manner according to unique characteristics of the T-cell clone and defines a unique binding site known as an idiotype of the T-cell clone. Conversely, the C domain does not participate in antigen binding.

The T-cell receptor according to the present invention may include one or more TCR α and/or TCR β variable domains. The variable domain may include a TCR α variable domain and a TCR β variable domain. The T-cell receptor according to the present invention may include one or more TCR α and/or TCR β constant domains.

The T-cell receptor according to the present invention may include a first polypeptide including variable and constant domains of a TCR α and/or a second polypeptide including variable and constant domains of a TCR β chain. For example, the T-cell receptor may be a αβ heterodimer or may be in the form of a single chain. An αβ TCR may include, for example, a full-length chain with both a cytoplasmic domain and a transmembrane domain. In some cases, there may be disulfide bonds introduced between residues of the constant domains.

The T-cell receptor according to the present invention is a disulfide-linked membrane-anchored heterodimeric protein consisting of highly variable alpha (α) and beta (β) chains that bind to a constant CD3 chain molecule to form a fully functional TCR. The α-β heterodimeric TCR has one α chain and one β chain. Each chain includes variable, optionally binding and constant regions, and the β chain also usually includes a short diversity region between the variable and binding regions, but the diversity region is often considered as a part of the binding region. Each variable region includes three complementarity determining regions (CDRs) embedded in a framework sequence, and one thereof is a hypervariable region defined as CDR3. The variable region includes several types of α chain variable (Vα) regions, and several types of β chain variable (Vβ) regions, and is distinguished by CDR1 and CDR2 and/or CDR3 sequences. CDR1 to CDR3 of the α chain variable (Vα) region are represented as CDR1α, CDR2α, and CDR3α, respectively, and CDR1 to CDR3 of the β chain variable (Vβ) region are represented as CDR1β, CDR2β, and CDR3β, respectively. In the international ImMunoGeneTics information System® (IMGT) nomenclature, the Vα types are referred to as unique TRAV numbers, and the Vβ types are referred to as unique TRBV numbers.

The T-cell receptor according to the present invention is a αβTCR, and extracellular portions of the αβTCR each consists of two polypeptides, and each extracellular portion has a membrane-proximal constant domain and a membrane-distal variable domain. Each of the constant and variable domains includes intra-chain disulfide bonds. The variable domain includes a highly polymorphic loop homology with the complementarity determining regions (CDRs) of the antibody.

The T-cell receptor of the present invention includes at least one CDR3 selected from the group consisting of SEQ ID NOs: 2 to 13. Specifically, the present invention may include α-chain CDR3s of SEQ ID NOs: 3, 5, 8, 9, and 13 or β-chain CDR3s of SEQ ID NOs: 2, 4, 6, 7, 10, 11, and 12.

More specifically, the T-cell receptor of the present invention may include:
CDR3α of SEQ ID NO: 3 and CDR3β of SEQ ID NO: 4;
CDR3α of SEQ ID NO: 5 and CDR3β of SEQ ID NO: 6;
CDR3α of SEQ ID NO: 1 and CDR3β of SEQ ID NO: 7;
CDR3α of SEQ ID NO: 8 and CDR3β of SEQ ID NO: 2;
CDR3α of SEQ ID NO: 9 and CDR3β of SEQ ID NO: 10;
CDR3α of SEQ ID NO: 3 and CDR3β of SEQ ID NO: 11;
CDR3α of SEQ ID NO: 3 and CDR3β of SEQ ID NO: 12; or
CDR3α of SEQ ID NO: 13 and CDR3β of SEQ ID NO: 4.

The T-cell receptor according to the present invention may include an α chain and a β chain including a membrane-proximal constant domain and a membrane-distal variable domain.

The T-cell receptor according to the present invention may include an α chain selected from the group consisting of SEQ ID NOs: 14, 16, 18, 20, 22, 24, 26, 28, and 30. The T-cell receptor according to the present invention may include a β chain selected from the group consisting of SEQ ID NOs: 15, 17, 19, 21, 23, 25, 27, 29, and 31.

Specifically, the T-cell receptor according to the present invention may include the following α chain and β chain:
α chain of SEQ ID NO: 14 and β chain of SEQ ID NO: 15;
α chain of SEQ ID NO: 16 and β chain of SEQ ID NO: 17;
α chain of SEQ ID NO: 18 and β chain of SEQ ID NO: 19;
α chain of SEQ ID NO: 20 and β chain of SEQ ID NO: 21;
α chain of SEQ ID NO: 22 and β chain of SEQ ID NO: 23;
α chain of SEQ ID NO: 24 and β chain of SEQ ID NO: 25;
α chain of SEQ ID NO: 26 and β chain of SEQ ID NO: 27;
α chain of SEQ ID NO: 28 and β chain of SEQ ID NO: 29; or
α chain of SEQ ID NO: 30 and β chain of SEQ ID NO: 31.

In some cases, the T-cell receptor according to the present invention may also be included in the form of a single chain. The TCR chain may include a first polypeptide α chain and a second polypeptide β chain. The α chain and β chain may include:
α chain of SEQ ID NO: 14 and β chain of SEQ ID NO: 15;
α chain of SEQ ID NO: 16 and β chain of SEQ ID NO: 17;
α chain of SEQ ID NO: 18 and β chain of SEQ ID NO: 19;
α chain of SEQ ID NO: 20 and β chain of SEQ ID NO: 21;
α chain of SEQ ID NO: 22 and β chain of SEQ ID NO: 23;
α chain of SEQ ID NO: 24 and β chain of SEQ ID NO: 25;
α chain of SEQ ID NO: 26 and β chain of SEQ ID NO: 27;
α chain of SEQ ID NO: 28 and β chain of SEQ ID NO: 29; or
α chain of SEQ ID NO: 30 and β chain of SEQ ID NO: 31.

The single chain may optionally include one or more linkers linking two or more polypeptides together. The linker may be, for example, a peptide. The linker may be a peptide linker and have a length of about 10 to 25 aa. For example, hydrophilic amino acids such as glycine and/or serine may be included, but are not limited thereto.

Specifically, the linker may include, for example, (GS)ₙ, (GGS)ₙ, (GSGGS)ₙ, or (GnS)ₘ (n and m are each 1 to 10), but the linker may be, for example, (GnS)ₘ (n and m are each 1 to 10). Specifically, the linker may include GGGGS.

The T-cell receptor of the present invention may include not only a sequence of the T-cell receptors described herein, but also biological equivalents thereof within a range capable of specifically recognizing MHC class I related protein (MR1). For example, additional changes may be made to the amino acid sequence to further improve the binding affinity and/or other biological properties of the T-cell receptor. Such modifications include, for example, deletion, insertion, and/or substitution of amino acid sequence residues. These amino acid variants are made based on the relative similarity of amino acid side-chain substituents, such as hydrophobicity, hydrophilicity, charges, sizes, and the like. By analysis of the size, shape, and type of the amino acid side-chain substituent, it can be seen that arginine, lysine, and histidine are all positively charged residues; alanine, glycine, and serine have similar sizes; and phenylalanine, tryptophan, and tyrosine have similar shapes. Accordingly, based on these considerations, arginine, lysine, and histidine; alanine, glycine, and serine; and phenylalanine, tryptophan, and tyrosine may be biologically functional equivalents.

Considering the variants having the above-described biologically equivalent activity, the T-cell receptor of the present invention is interpreted to include a sequence representing substantial identity with the sequence described in SEQ ID NO. The substantial identity means a sequence exhibiting homology of at least 90%, most preferably homology of at least 95%, homology of 96% or more, 97% or more, 98% or more, and 99% or more, when the sequence of the present invention is aligned to correspond to any other sequence as much as possible and the aligned sequence is analyzed using an algorithm commonly used in the art. Alignment methods for sequence comparison are known in the art. An NCBI Basic Local Alignment Search Tool (BLAST) is accessible from NBCI, etc., and may be used in conjunction with sequence analysis programs such as blastp, blasm, blastx, tblastn, and tblastx on the Internet. BLAST is accessible at www.ncbi.nlm.nih.gov/BLAST/. A sequence homology comparison method using these programs can be found at www.ncbi.nlm.nih.gov/B LAST/blast_help.html.

Based thereon, the T-cell receptor of the present invention may have homology of 90%, 91%, 92%, 93%, 94%, 95%, 96%, 97%, 98%, 99%, or 99% or more with the specified sequence or the entirety described the specification. Such homology may be determined by sequence comparison and/or alignment by methods known in the art. For example, the percent sequence homology of proteins according to the present invention may be determined using a sequence comparison algorithm (i.e., BLAST or BLAST 2.0), manual alignment, or visual inspection.

The T-cell receptor according to the present invention is a T-cell receptor protein that binds to a non-polymorphic MHC I-related MR1 antigen-presenting molecule, which is expressed on tumor or cancer cells and binds to an MR1 molecule that presents a tumor- or cancer-related antigen. In the present invention, the cell including the T-cell receptor binding to the MR1 molecule is also referred to as an MR1-restricted T-cell.

The T-cell receptor according to the present invention may be used to specifically recognize MR1-expressing tumor or cancer cells for T cells for tumor or cancer treatment. Upon contact with the MR1-expressing tumor or cancer cells (which present a tumor or cancer antigen in an MR1-restricted manner), the T-cell receptor is activated to exhibit reactivity.

In another aspect, the present invention relates to a nucleic acid encoding the T-cell receptor. The T-cell receptor may be produced in a recombinant manner by isolating the nucleic acids encoding the T-cell receptor of the present invention.

The “nucleic acid” has a meaning of comprehensively including DNA (gDNA and cDNA) and RNA molecules, and nucleotides, which are basic structural units in the nucleic acid, are not only natural nucleotides, but also analogues with modified sugar or base moieties. A sequence of a nucleic acid encoding heavy and light chain variable regions of the present invention may be modified. The modification includes addition, deletion, or non-conservative or conservative substitution of nucleotides.

Based thereon, the T-cell receptor of the present invention may have homology of 90%, 91%, 92%, 93%, 94%, 95%, 96%, 97%, 98%, 99%, or 99%, or more with the specified sequence or the entirety described the specification. Such homology may be determined by sequence comparison and/or alignment by methods known in the art. For example, the percent sequence homology of nucleic acids or proteins according to the present invention may be determined using a sequence comparison algorithm (i.e., BLAST or BLAST 2.0), manual alignment, or visual inspection.

The nucleic acid encoding the T-cell receptor may include a nucleic acid selected from the group consisting of SEQ ID NOs: 32 to 49.

In a specific embodiment according to the present invention, the T-cell receptor may include:
an α chain-coding nucleic acid of SEQ ID NO: 32 and a β chain-coding nucleic acid of SEQ ID NO: 33;
an α chain-coding nucleic acid of SEQ ID NO: 34 and a β chain-coding nucleic acid of SEQ ID NO: 35;
an α chain-coding nucleic acid of SEQ ID NO: 36 and a β chain-coding nucleic acid of SEQ ID NO: 37;
an α chain-coding nucleic acid of SEQ ID NO: 38 and a β chain-coding nucleic acid of SEQ ID NO: 39;
an α chain-coding nucleic acid of SEQ ID NO: 40 and a β chain-coding nucleic acid of SEQ ID NO: 41;
an α chain-coding nucleic acid of SEQ ID NO: 42 and a β chain-coding nucleic acid of SEQ ID NO: 43;
an α chain-coding nucleic acid of SEQ ID NO: 44 and a β chain-coding nucleic acid of SEQ ID NO: 45;
an α chain-coding nucleic acid of SEQ ID NO: 46 and a β chain-coding nucleic acid of SEQ ID NO: 47; or
an α chain-coding nucleic acid of SEQ ID NO: 48 and a β chain-coding nucleic acid of SEQ ID NO: 49.

The DNA encoding the T-cell receptor may be easily isolated or synthesized using conventional molecular biological techniques (e.g., by using an oligonucleotide probe capable of specifically binding to DNA encoding the T-cell receptor), and the nucleic acid is isolated and inserted into a replicable vector to be additionally cloned (DNA-amplified) or expressed. Based thereon, another aspect of the present invention relates to a recombinant vector including the nucleic acid.

As used herein, the term “vector” refers to a nucleic acid molecule capable of transporting another nucleic acid linked as the nucleic acid molecule. For example, the vector includes a single-stranded, double-stranded, or partially double-stranded nucleic acid molecule; a nucleic acid molecule including one or more free ends and non-free ends (e.g., circular); a nucleic acid molecule including DNA, RNA or both; and various other polynucleotides known in the art.

For example, the vector is a “plasmid,” which refers to a circular double-stranded DNA loop, and an additional DNA segment may be inserted thereto by, for example, a standard molecular cloning technique.

For example, the vector may include a viral vector. The viral vector may include, for example, a Lentiviral Vector (LV) or a Retroviral Vector (RV).

The LV includes Retrovirus ssRNA as a genetic substance and has a packaging capacity of about 8 kb. Through LV, foreign genes may be transfected into dividing cells without dilution. The LV can transfect both dividing cells and non-dividing cells. The LV includes a Transfer Vector, a Packaging Vector, and an Envelope Vector, and the three vectors are co-transfected to generate virus particles, and then the virus particles are quantitative-purified to select cells in which a target gene is well delivered.

The transfer vector may include Tat (transcription induction protein for gene expression) binding sites 5′ LTR and 3′ LTR, a packaging signal (ψ), and a transgene. The packaging vector may include a viral structural gene such as gag and/or pol in which a replication-deleted packaging signal (Δψ) is deleted and enzymes such as capsid, reverse transcriptase, protease, and integrase are expressed. The packaging vector may include a viral regulatory gene (tat and/or rev) such as Tat for inducing transcription and/or Rev for transporting mRNA. The envelope vector may include a viral envelope expression gene, viral env.

In relation to RV, replication is initiated through provirus DNA, in which RNA having genetic information is converted to double-stranded DNA by reverse transcriptase. External gene introduction of about 8 kb is possible. Viral particles may be produced by introducing a plasmid into which a therapeutic gene containing LTR has been introduced into a virus particular cell line. The cell line supplies gag, pol, and env proteins.

The cell line includes a Transfer Vector, a Packaging Vector, and an Envelope Vector, and the three vectors are co-transfected to generate virus particles, and then the virus particles are quantitative-purified to select cells in which a target gene is well delivered.

The transfer vector may include Tat (transcription induction protein for gene expression) binding sites 5′ LTR and 3′ LTR, and a transgene. Through 5′ LTR and 3′ LTR, viral replication, insertion into a host, and viral gene expression are induced. The packaging vector may include a viral structural gene such as gag and/or pol in which a replication-deleted packaging signal (Δψ) is deleted and enzymes such as capsid, reverse transcriptase, protease, and integrase are expressed. The packaging vector may include a viral envelope expression gene, viral env.

In the case of the viral vector, a virus-derived DNA or RNA sequence is present in a vector for packaging into a virus (e.g., retrovirus, replication defective retrovirus, adenovirus, replication defective adenovirus, and adeno-associated virus). The viral vector includes polynucleotides carried by a virus for transfection into a host cell.

In some cases, the vector may be autonomously replicated in a host cell to be introduced (e.g., bacterial vector having bacterial replicating Ori and episomal mammalian vector). Other vectors (e.g., non-episomal mammalian vectors) are integrated into a genome of the host cell when being introduced into the host cell to be replicated with the host genome.

A specific vector may indicate the expression of genes which are operably linked. Such a vector is referred to as an “expression vector” in the present invention. Common expression vectors useful in recombinant DNA technology are often in the form of plasmids.

A recombinant expression vector may include a nucleic acid in a form suitable for expression of the nucleic acid in a host cell, which means including one or more regulatory elements that may be selected on the basis of the host cell so that the recombinant expression vector is used for expression, that is, operably linked to a nucleic acid sequence to be expressed.

In the recombinant expression vector, the “operably linked” means that a nucleotide sequence of interest is linked to regulatory elements in a manner of allowing the expression of the nucleotide sequence (e.g., in an in vitro transcription/translation system or in a host cell when the vector is introduced into the host cell).

The “regulatory element” may include a promoter, an enhancer, an internal ribosome entry site (IRES), and other expression control elements (e.g., transcription termination signals such as a polyadenylation signal and a poly-U sequence). The regulatory element includes an element that instructs induced or constitutive expression of a nucleotide sequence in many types of host cells, and an element (e.g., tissue-specific regulatory sequence) that instructs expression of a nucleotide sequence only in a specific host cell. The tissue-specific promoter may instruct the expression primarily in a desired tissue of interest such as muscle, neuron, bone, skin, blood, a specific organ (e.g., liver, pancreas), or a specific cell type (e.g., lymphocyte). The regulatory element may also instruct the expression in a temporally-dependent manner, such as in a cell-cycle-dependent or developmental stage-dependent manner, which may or not be tissue or cell-type specific.

In some cases, the vector includes one or more pol III promoters, one or more pol II promoters, one or more pol I promoters, or combinations thereof. Examples of the pol III promoters include U6 and H1 promoters without limitation. Examples of the pol II promoters include a retroviral Rous Sarcoma Virus (RSV) LTR promoter (optionally with an RSV enhancer), a cytomegalovirus (CMV) promoter (optionally with a CMV enhancer) (e.g., Boshart et al. al (1985) Cell 41:521-530), an SV40 promoter, a dihydrofolate reductase promoter, a β-actin promoter, a phosphoglycerol kinase (PGK) promoter, and an EF1α promoter without limitation.

The “regulatory element” may include enhancers, such as WPRE; CMV enhancer; R-U5′ segment in LTR of HTLV-I; SV40 enhancer; and an intronic sequence between exons 2 and 3 of rabbit β-globin. It will be appreciated by those skilled in the art that a design of the expression vector may depend on factors such as the selection of a host cell to be transformed, a desired expression level, and the like. The vector may be introduced into a host cell to generate a transcript, a protein, or a peptide including a fusion protein or peptide encoded by the nucleic acid as described herein (e.g., clustered regularly interspaced short palindromic repeats (CRISPR) transcripts, proteins, enzymes, mutants thereof, fusion proteins thereof, etc.). Useful vectors include lentivirus and adeno-associated virus, and these types of vectors may also be selected to target a specific type of cell.

The “Polynucleotide”, “nucleotide”, “nucleotide sequence”, “nucleic acid”, and “oligonucleotide” are used interchangeably. A polymeric form of nucleotides having any length, deoxyribonucleotide or ribonucleotide, or analogues thereof may be included. The polynucleotide may have any three-dimensional structure and may perform any known or unknown function. The polynucleotide may include one or more modified nucleotides, such as methylated nucleotides and nucleotide analogues. Modifications for the nucleotide structure may be possible before or after assembly of the polymer.

The vector may be designed for expression of endonucleases (e.g., nucleic acid transcripts, proteins, or enzymes) and cleavage factors according to the present invention in either prokaryotic or eukaryotic cells. For example, endonuclease and cleavage factor transcripts may be expressed in bacterial cells such as E. coli, insect cells (using a baculovirus expression vector), yeast cells, or mammalian cells. In some cases, the recombinant expression vector may be transcribed and translated in vitro using, for example, a T7 promoter regulatory sequence and T7 polymerase.

The vector may be introduced and proliferated in prokaryotes. In some embodiments, the prokaryotes may be used to amplify copies of vectors to be introduced into eukaryotic cells or as an intermediate vector in the production of vectors to be introduced into eukaryotic cells (e.g., amplifying a plasmid as part of a viral vector packaging system). The prokaryotes may be used to amplify copies of the vector and express one or more nucleic acids, and to provide a source of one or more proteins for delivery, for example, into a host cell or host organism. The expression of the proteins in the prokaryotes may be performed in E. coli with a vector, including a constitutive or induced promoter.

The vector may be delivered in vivo or into cells through electroporation, lipofection, viral vectors, nanoparticles, and protein translocation domain (PTD) fusion protein methods, respectively.

Components of the vector generally include, but are not limited to, one or more of the following: signal sequences, origins of replication, one or more marker genes, enhancer elements, promoters, transcription termination sequences, etc. The nucleic acid encoding the T-cell receptor is operably linked, such as promoters and transcription termination sequences.

The “operably linked” means a functional linkage between a nucleic acid expression regulatory sequence (e.g., a promoter, signal sequence, or array of transcriptional regulator binding sites) and the other nucleic acid sequence, so that the regulatory sequence regulates the transcription and/or translation of the other nucleic acid sequence.

In another aspect, the present invention relates to a T cell expressing the T-cell receptor.

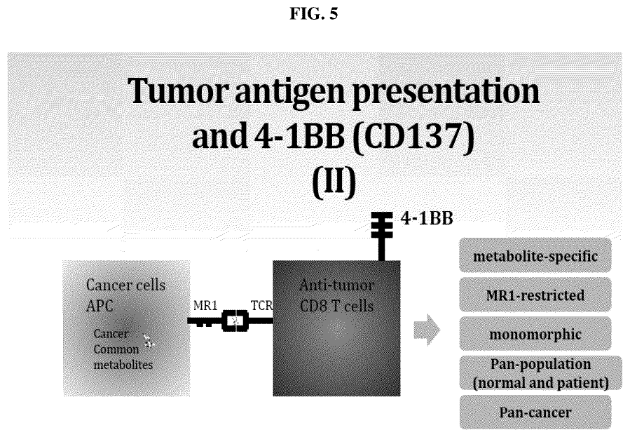

The T cell may be a cultured T cell, for example, all T cells such as a primary T cell or a cultured cell line, for example, a T cell derived from Jurkat, SupT1, etc., or a T cell obtained from a mammal, preferably a T cell or T cell precursor from a human patient. When obtained from the mammal, the T cell may be obtained from a plurality of sources, including blood, bone marrow, lymph nodes, thymus, or other tissues or fluids, but is not limited thereto. The T cell may also be supplemented or purified. Preferably, the T cell is a human T cell. More preferably, the T cell is a T cell isolated from the human. The T cell may be selected from the group consisting of a CD4+ T cell; a CD8+ cytotoxic T lymphocyte (CTL); a gamma-delta T cell; and a T cell isolated from a tumor infiltrating lymphocyte (TIL) and a peripheral blood mononuclear cell (PBMC), but is not limited thereto. The T cell may be a non-limiting type of T cell and may be a non-limiting development stage, and may include CD4+ and/or CD8+, CD4− helper T cells such as Th1 and Th2 cells, CD8+ T cells (e.g., cytotoxic T cells), tumor infiltrating cells (TIL), memory T cells, natural T cells, and the like, but is not limited thereto. Preferably, the T cell may be a CD8+ T cell. A specific structure of the CD8+ T cell according to the present invention is illustrated in FIG. 5.

The T cells are lymphocytes, specifically human T lymphocytes, and may be preferably T lymphocytes such as CD4+ or CD8+ T cells. The T cells may be tumor or cancer reactive T cells specific to tumor or cancer cells.

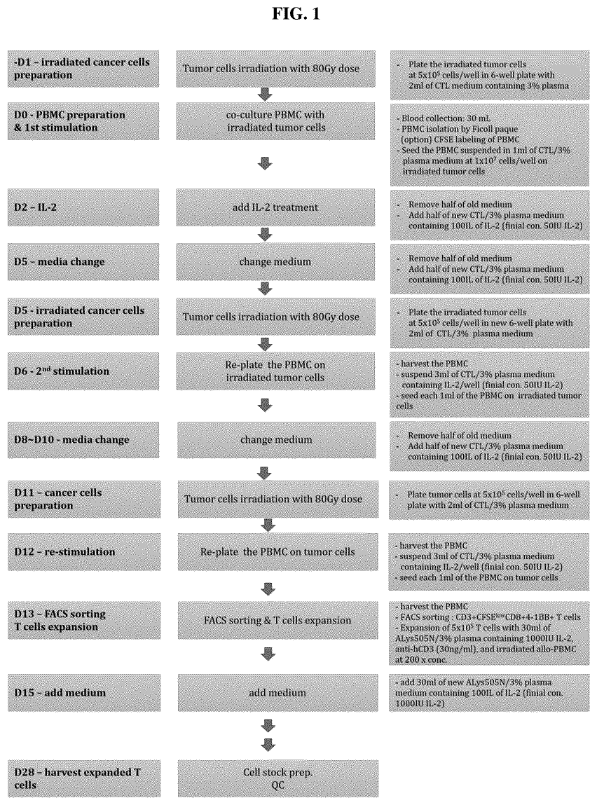

According to the present invention, MR1-restricted cancer killing CD8+T lymphocytes may be provided. A specific method for isolating and mass-culturing the MR1-restricted cancer killing CD8+T lymphocytes is illustrated in FIG. 1.

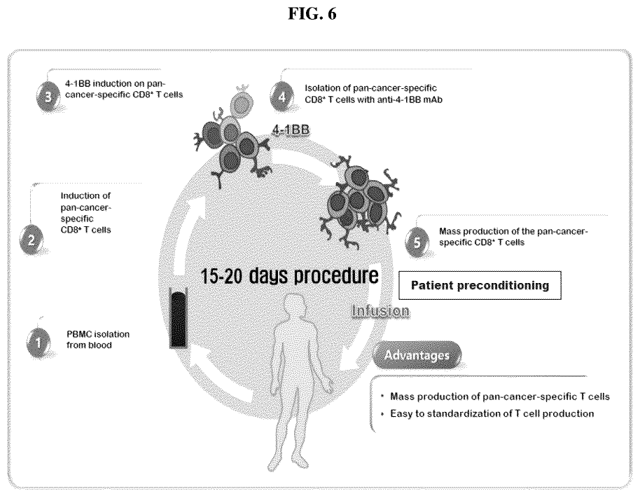

In addition, a detailed schematic diagram of a platform technology for producing CD8+ T cells having pan-cancer killing ability according to the present invention is illustrated in FIG. 6.

In another aspect, the present invention relates to an anti-tumor or anti-cancer composition including the T-cell receptor, the nucleic acid, the vector, or the T cell.

In the present invention, the “cancer” and “tumor” are used in the same meaning and refer to or mean a mammalian physiological condition typically characterized by unregulated cell growth/proliferation.

Cancers or cancer types that may be treated with the composition of the present invention are not particularly limited, and includes both solid cancer and blood cancer. For example, the cancer or cancer type may include any one selected from the group consisting of acute lymphocytic cancer, acute myelogenous leukemia, alveolar rhabdomyosarcoma, bone cancer, brain cancer, breast cancer, anal cancer, anal, anal canal or rectoanal cancer, eye cancer, cancer of the intrahepatic bile duct, cancer of the joints, cancer of the neck, bladder or pleura, cancer of the nose, cancer of nasal cavity or middle ear, cancer of the oral cavity, cancer of the vagina, cancer of the vulva, chronic lymphocytic leukemia, chronic bone marrow cancer, colon cancer, esophageal cancer, cervical cancer, gastrointestinal carcinoid tumor, glioma, Hodgkin's lymphoma, hypopharyngeal cancer, kidney cancer, laryngeal cancer, liver cancer, lung cancer, malignant mesothelioma, melanoma, multiple myeloma, nasopharyngeal cancer, non-Hodgkin's lymphoma, cancer of the oropharynx, ovarian cancer, cancer of the penis, pancreatic cancer, peritoneal, serous and mesenteric cancer, pharynx cancer, prostate cancer, rectal cancer, kidney cancer, skin cancer, small intestine cancer, soft tissue cancer, stomach cancer, testicular cancer, thyroid cancer, cancer of the uterus, ureteral cancer, and bladder cancer, but is not limited thereto.

The therapeutic composition of the present invention is a composition for the prevention or treatment of cancer, and in the present invention, the term “prevention” refers to any action that suppresses cancer or delays the progression of cancer by administering the composition of the present invention, and the “treatment” means suppression of cancer development, and relief or elimination of symptoms.

In the composition, it is preferable that the number of T cells expressing the T-cell receptor is 0.1 to 30 times, specifically 0.2 to 25 times, more specifically 0.25 times to 20 times greater than the number of tumor cells in a treated subject, but it is not limited thereto.

The composition may additionally include a pharmaceutically acceptable excipient. Examples of the excipient may include surfactants, preferably polysorbate-based nonionic surfactants; buffers such as neutral buffered saline and human salt buffered saline; sugars or sugar alcohols such as glucose, mannose, sucrose or dextran, and mannitol; amino acids such as glycine and histidine, proteins or polypeptides; antioxidants; chelating agents such as EDTA or glutathione; penetrants; adjuvants; and preservatives, but are not limited thereto.

The composition of the present invention may be formulated by using a method known in the art so as to provide rapid, sustained, or delayed release of active ingredients after being administrated to mammals except for the human. The formulation may be powders, granules, tablets, emulsions, syrups, aerosols, soft or hard gelatin capsules, sterile injectable solutions, and sterile powder forms.

The pharmaceutical composition may be various oral or parenteral formulations. When the pharmaceutical composition is formulated, the formulation may be prepared by using diluents or excipients, such as a filler, an extender, a binder, a wetting agent, a disintegrating agent, and a surfactant, which are generally used. Solid formulations for oral administration include tablets, pills, powders, granules, capsules, and the like, and these solid formulations may be prepared by mixing at least one excipient, for example, starch, calcium carbonate, sucrose or lactose, gelatin, and the like with at least one compound. Further, lubricants such as magnesium stearate, talc, and the like may be used in addition to simple excipients. Liquid formulations for oral administration may correspond to a suspension, an oral liquid, an emulsion, a syrup, and the like, and may include various excipients, for example, a wetting agent, a sweetener, an aromatic agent, a preserving agent, and the like, in addition to water and liquid paraffin which are commonly used as simple diluents. Formulations for parenteral administration include a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The present invention relates to a method for treating tumor or cancer including administering the T-cell receptor, the nucleic acid, the vector, or the T cell to a subject. The present invention also relates to a use of the T-cell receptor, the nucleic acid, the vector, or the T cell for the treatment of tumors or cancer. The present invention further relates to a use of the T-cell receptor, the nucleic acid, the vector, or the T cell for preparing drugs for treatment of tumors or cancer.

The subject may be mammals having tumors, specifically humans, but is not limited thereto.

The composition may be administered through oral administration, infusion, intravenous injection, intramuscular injection, subcutaneous injection, intraperitoneal injection, intrarectal administration, topical administration, intranasal injection, etc., but is not limited thereto.

The dose of the active ingredients may be appropriately selected according to various factors such as a route of administration, age, sex, weight and severity of a patient, and the composition may be administered concurrently with known compounds having an effect of preventing, improving or treating tumor or cancer symptoms.

Hereinafter, the present invention will be described in more detail through Examples. These Examples are just illustrative of the present invention, and it will be apparent to those skilled in the art that it is not interpreted that the scope of the present invention is limited to these Examples.

### Example 1. Isolation of MR1-Restricted T Cells

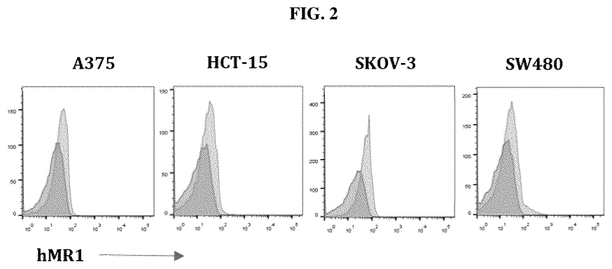

It was confirmed that MR1 was expressed at a low level in human melanoma (A375), breast cancer (SKOV-3), colorectal cancer cell lines (SW480, HCT-15), and the like (FIG. 2).

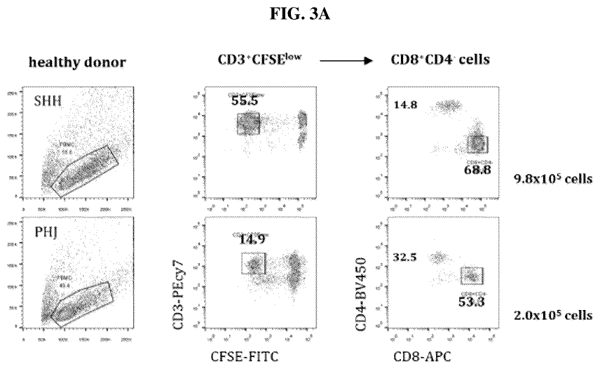

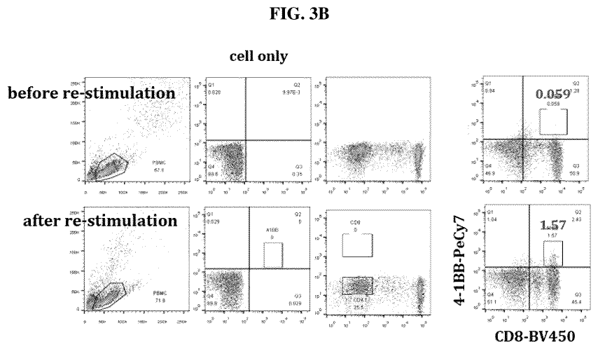

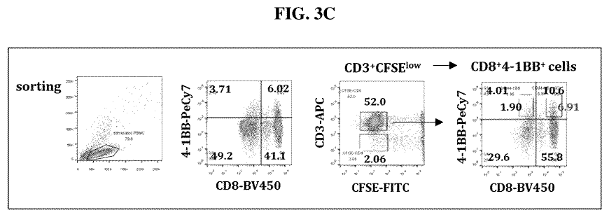

MR1-restricted T cells were isolated and proliferated based on a proliferation dye. PBMCs from two healthy donors were co-cultured and stimulated with irradiated SW480 cells, and then CD8+CD4− T cells were isolated from gated CD3+CFSElow cells. The isolated CD3⁺CD8⁺CFSE^{low} cells were massively proliferated using a rapid expansion method (FIG. 3A).

PBMCs from healthy donors were co-cultured with irradiated SW480 cells. 4-1BB expression was confirmed in MR1-restricted CD8+ T cells before and after re-stimulation with the irradiated SW480 cells. No expression of 4-1BB was detected in CD8+ T cells before re-stimulation with the SW480 cells, but 4-1BB expression was detected after re-stimulation (FIG. 3B).

The PBMCs from healthy donors were co-cultured and stimulated with the irradiated SW480 cells, and then 4-1BB⁺CD8⁺ T cells were isolated from the gated CD3⁺CFSElow cells. The isolated 4-1BB⁺CD8⁺ T cells were massively proliferated using a rapid expansion method (FIG. 3C).

### Example 2. MR1-Restricted T Cells Recognized Cancer Different from MAIT Cells

Mucosal-associated invariant T (MAIT) cells consisted of about 1 to 8% of peripheral blood T cells and about 40% of T cells present in mucosal tissues, mesenteric lymph nodes, and liver, and were known to recognize nonpeptidic Ag through MR1. The antigens recognized by the MAIT cells were riboflavin-derivatives, particularly 5-(2-oxopropylideneamino)-6-d-ribitylaminouracil (5-OP-RU) produced by bacteria and fungi, and had a TCR Vα 7.2⁺ CD161^{high} phenotype.

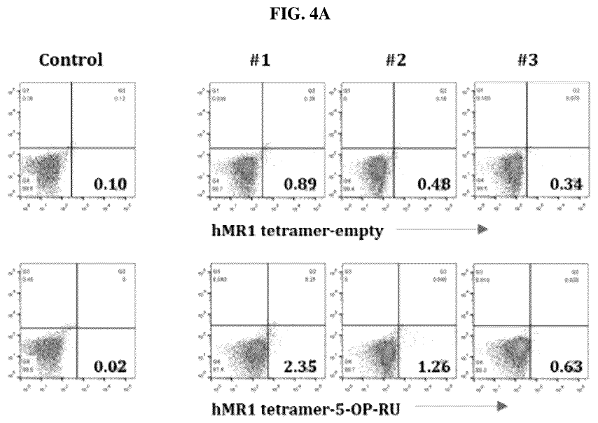

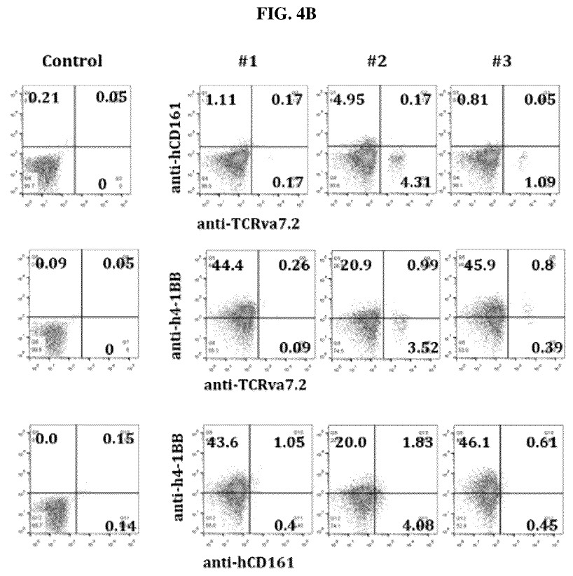

In addition to the MR1-restricted T cells stimulated by the SW480 cells, the MAIT cells were stained with MR1 tetramer-empty and MR1 tetramer-loaded 5-OP-RU. After being cultured and stimulated with the SW480 cells, when the isolated MR1-restricted T cells were stained with 5-OP-RU tetramer, an MR1 ligand for the MAIT cells, the MR1-restricted T cells did not bind to the 5-OP-RU tetramer (FIG. 4A). As a result, it was confirmed that the selected MR1-restricted T cells were not the MALT cells.

The phenotype for TCRVα7.2+CD161^{high} by the MR1-restricted T cells stimulated by the SW480 cells was analyzed. After being cultured and stimulated with the SW480 cells, it was confirmed that TCRVα7.2 and CD161 were not double-stained in the isolated MR1-restricted T cells, so that the selected MR1-restricted T cells were not the MALT cells. In addition, 4-1BB expression was confirmed in the MR1-restricted T cells of Vα7.2-CD161− fraction (FIG. 4B).

### Example 3. Confirmation of Cancer-Killing Ability of TCR-Expressing T Cells

3.1 Construction of Lentiviral Transfection Plasmid

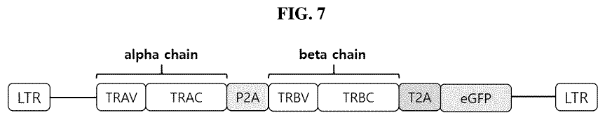

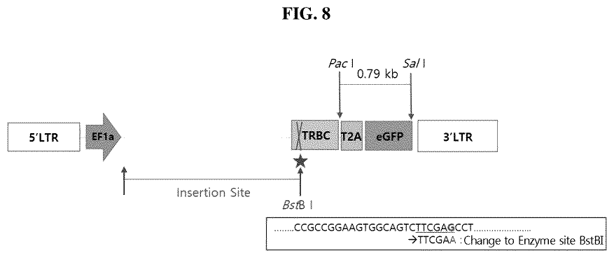

Lentiviral transfection plasmid cloning was performed for preparation of MR1 TCR-T cells. A structure of the lentiviral transfection plasmid constructed for MR1 TCR-T cells is illustrated in FIG. 7. A vector backbone for the lentiviral transfection plasmid cloning was constructed. The vector backbone structure used for the lentiviral transfection plasmid cloning for MR1 TCR expression is illustrated in FIG. 8. The vector backbone structure was a structure capable of sequentially expressing T2A, P2A self-cleavage peptides, and eGFP in addition to TRAC and TRBC genes corresponding to constant regions of an α chain and a β chain, respectively.

3.2 Gene Synthesis

Genes were synthesized based on α chain and β chain sequence information for MR1 TCR clones. The gene sequence information is shown in Table 1 below.

TABLE 1

| ID | Clone Name | Chain | V | J | C |
|---|---|---|---|---|---|
| MC.7.G5 | MC.7.GS | α | TRAV38-2/DV8 | TRAJ31 | TRAC |
| | | β | TRBV25-1 | TRBJ2-3 | TRBC2 |
| EUMR1-03 | BSK2_2 | α | TRAV5 | TRAJ15 | TRAC |
| | | β | TRBV9 | TRBJ2-5 | TRBC2 |
| EUMR1-14 | MHH2_4 | α | TRAV21 | TRAJ48 | TRAC |
| | | β | TRBV20-1 | TRBJ1-5 | TRBC1 |
| EUMR1-31 | MC.7.G5 alpha | α | TRAV38-2 | DV8_TRAJ31 | TRAC |
| | MR1011 beta | β | TRBV25-1 | TRBJ2-3 | TRBC2 |
| EUMR1-32 | MR1011 alpha | α | TRAV38-2 | DVB_TRAJ31 | TRAC |
| | MC.7.G5 beta | β | TRBV25-1 | TRBJ2-3 | TRBC2 |
| EUMR1-33 | Basel_1 | α | TRAV29DV5 | TRAJ23 | TRAC |
| | | β | TRBV28 | | TRBC2 |
| EUMR1-36 | MR1002-2-like_1 | α | TRAV5 | TRAJ15 | TRAC |
| | | β | TRBV9 | TRBJ2-5 | TRBC2 |
| EUMR1-37 | MR1002-2-like_2 | α | TRAV5 | TRAJ15 | TRAC |
| | | β | TRBV6-2 | TRBJ2-5 | TRBC2 |
| EUMR1-38 | MR1002-2-like_3 | α | TRAV5 | TRAJ15 | TRAC |
| | | β | TRBV9 | TRBJ2-5 | TRBC2 |

TABLE 2

| ID | Clone Name | Chain | CDR3 aa | no. |
|---|---|---|---|---|
| MC.7.G5 | MC.7.G5 | α | CAYRSAVNARLMF | 1 |
| | | β | CASSEARGLAEFTDTQYF | 2 |
| EUMR1-03 | BSK2_2 | α | CAEIKRTALIF | 3 |
| | | β | CASGSGIRQETQYF | 4 |
| EUMR1-14 | MHH2.4 | α | CAVPSNFGNEKLTF | 5 |
| | | β | CSARALYTGEDSNQPQHF | 6 |
| EUMR1-31 | MC.7.G5 alpha | α | CAYRSAVNARLMF | 1 |
| | MR1011 beta | β | CASRGLAEFTDTQYF | 7 |
| EUMR1-32 | MR1011 alpha | α | CAYRSALNARLMF | 8 |
| | MC.7.G5 beta | β | CASSEARGLAEFTDTQYF | 2 |
| EUMR1-33 | Basel_1 | α | CAAQIYNQGGKLIFGQG | 9 |
| | | β | CASSFSSGKQYFGPGT | 10 |
| EUMR1-36 | MR1002-2- | α | CAEIKRTALIF | 3 |
| | like_1 | β | CASSGSGEQETQYF | 11 |
| EUMR1-37 | MR1002-2- | α | CAEIKRTALIF | 3 |
| | like_2 | β | CASSSGSSGVYQETQYF | 12 |
| EUMR1-38 | MR1002-2- | α | CAELAGTALIF | 13 |
| | like_3 | β | CASGSGIRQETQYF | 4 |

TABLE 3

| ID | Type | Sequence |
|---|---|---|
| MC.7.G5 | Nucleotide | [α chain] (SEQ ID No. 32) |
| | | ATGGCTTGTCCAGGTTTTTTGTGGGCGCTGGTCATCTCCACTTGCCTCGA |
| | | ATTTAGTATGGCCCAGACGGTGACGCAGTCACAGCCGGAGATGTCCGTT |
| | | CAAGAAGCTGAAACTGTCACACTTAGTTGTACGTATGACACCTCAGAAA |
| | | GCGACTATTACTTGTTTTGGTATAAACAACCTCCCTCTCGCCAGATGATCC |
| | | TTGTTATACGACAAGAGGCATACAAACAGCAGAATGCAACTGAGAATC |
| | | GGTTTAGCGTAAACTTTCAAAAGGGGGCCAAAAGTTTCTCTCTGAAAAT |
| | | TTCTGACTCTCAATTGGGAGACGCCGCGATGTATTTCTGTGCGTATAGGA |
| | | GCGCCGTAAACGCTCGACTTATGTTTGGCGACGGAACTCAACTCGTAGT |
| | | CAAGCCCAACATACAAAATCCTGATCCGGCCGTCTACCAACTGAGGGAT |
| | | TCAAAGTCTTCCGACAAGTCTGTTTGTCTGTTTACGGATTTCGACAGCCA |
| | | AACGAACGTATCACAGTCCAAGGACTCCGATGTATATATCACCGATAAGA |
| | | CAGTATTGGATATGAGGTCCATGGATTTCAAAAGCAATTCCGCTGTGGCC |
| | | TGGTCAAATAAGAGCGACTTCGCCTGTGCGAATGCATTCAATAATTCTAT |
| | | AATCCCAGAGGACACGTTTTTTCCCAGCCCTGAGAGCAGTTGCGACGTT |
| | | AAGCTCGTTGAGAAATCCTTTGAGACGGATACAAATCTTAATTTCCAGA |
| | | ATCTTAGTGTAATAGGTTTCCGAATCCTGCTGCTTAAAGTAGCCGGTTTC |
| | | AACTTGCTGATGACTTTGAGACTTTGGTCCAGC |
| | | [P2A] (SEQ ID No. 50) |
| | | GGAAGGGGCGCTACGAACTTTAGTCTGCTCAAACAGGCTGGGGATGTA |
| | | GAGGAAAATCCGGGGCCT |
| | | [β chain] (SEQ ID No. 33) |
| | | ATGACGATAAGGCTTCTGTGCTATATGGGGTTTTATTTCCTTGGTGCAGG |
| | | CTTGATGGAGGCAGATATCTACCAAACTCCACGATATCTGGTCATTGGAA |
| | | CCGGGAAGAAGATTACATTGGAATGCTCCCAGACGATGGGCCATGACA |
| | | AGATGTACTGGTACCAACAAGATCCGGGAATGGAGCTCCACCTTATTCA |
| | | CTACTCCTACGGGGTGAACTCCACGGAGAAGGGGGACCTGTCTTCCGÅ |
| | | AAGTACAGTCAGCCGAATCAGGACCGAACATTTCCCCCTGACTCTGGAG |
| | | TCAGCGCGACCGTCTCATACAAGCCAATATCTTTGCGCCTCATCTGAAGC |
| | | GCGCGGATTGGCTGAGTTCACAGACACACAATATTTCGGCCCAGGTAC |
| | | AAGACTCACAGTTTTGGAGGACCTGAAGAACGTGTTCCCGCCGGAAGT |
| | | GGCAGTCTTCGAGCCTAGCGAGGCAGAAATAAGCCATACTCAGAAAGC |
| | | GACCCTGGTATGCCTGGCCACAGGATTCTATCCCGACCATGTCGAGCTC |
| | | AGCTGGTGGGTAAACGGTAAAGAGGTACATAGTGGTGTGTCAACAGAC |
| | | CCTCAGCCCCTTAAAGAGCAACCTGCCCTTAACGATAGTCGGTATTGCTT |
| | | GTCATCTAGGCTCAGAGTTTCCGCCACGTTTTGGCAGAATCCTAGGAAC |
| | | CATTTTCGCTGCCAGGTCCAATTCTACGGGCTCTCAGAAAACGACGAAT |
| | | GGACCCAGGACAGAGCGAAGCCAGTAACTCAGATAGTCAGCGCTGAA |
| | | GCATGGGGACGAGCGGATTGCGGATTTACGAGCGAGTCCTACCAGCAA |
| | | GGCGTTCTCTCCGCCACCATCCTTTACGAGATTTTGCTCGGGAAGGCCA |
| | | CATTGTACGCCGTATTGGTAAGTGCGCTTGTCCTGATGGCCATGGTCAAG |
| | | AGAAAAGATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 14) |
| | | MACPGFLWALVISTCLEFSMAQTVTQSQPEMSVQEAETVTLSCTYDTSES |
| | | DYYLFWYKQPPSRQMILVIRQEAYKQQNATENRFSVNFQKAAKSFSLKIS |
| | | DSQLGDAAMYF [symbol] GDGTQLVVKPNIQNPDPAVYQLRD |
| | | SKSSDKSVCLFTDFDSQTNVSQSKDSDVYITDKTVLDMRSMDFKSNSAV |
| | | AWSNKSDFACANAFNNSIIPEDTFFPSPESSCDVKLVEKSFETDINLNFQN |
| | | LSVIGFRILLLKVAGENLLMTLRLWSS |
| | | [P2A] (SEQ ID No. 53) |
| | | GSGATNFSLLKQAGDVEENPGP |
| | | [β chain) [symbol] (SEQ ID No. 15) |
| | | MTIRLLCYMGFYFLGAGLMEADIYQTPRYLVIGTGKKITLECSQTMGHDKM |
| | | YWYQQDPGMELHLIHYSYGVNSTEKGDLSSESTVSRIRTEHFPLTLESARP |
| | | SHTSQYL [symbol] GPGTRLTVLEDLKNVFPPEVAVFEPSE |
| | | AEISHTQKATLVCLATGFYPDHVELSWWVNGKEVHSGVSTDPQPLKEQP |
| | | ALNDSRYCLSSRLRVSATFWQNPRNHFRCQVQFYGLSENDEWTQDRAK |
| | | PVTQIVSAEAWGRADCGFTSESYQQGVLSATILYEILIGKATLYAVLVSALVL |
| | | MAMVKRKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGQGPVLLPDNH |
| | | YLSTQSALSKDPNEKRDHMVLLEFVTAAGITLGMDELYK* |
| EUMR1-03 | Nucleotide | [α chain] (SEQ ID No. 34) |
| | | ATGAAGACCTTCGCCGGCTTCAGCTTCCTGTTCCTGTGGCTGCAGCTGG |
| | | ACTGTATGAGCAGAGGCGAGGATGTGGAACAGAGCCTGTTTCTGAGCG |
| | | TCAGAGAAGGCGACAGCAGCGTGATCAACTGCACCTACACCGACAGCT |
| | | CCAGCACCTACCTGTACTGGTACAAGCAAGAACCTGGCGCCGGACTGC |
| | | AGCTGCTGACCTACATCTTCAGCAACATGGACATGAAGCAGGACCAGC |
| | | GGCTGACCGTGCTGCTGAACAAGAAGGATAAGCACCTGAGCCTGGGG |
| | | ATCGCCGATACACAGACAGGCGACTCCGCCATCTACTTTTGCGCCGAGA |
| | | TCAAGCGGACAGCCCTGATCTTTGGCAAGGGCACAACACTGAGCGTGT |
| | | CCAGCAACATTCAGAACCCCGATCCTGCCGTGTACCAGCTGAGAGACA |
| | | GCAAGAGCAGCGACAAGAGCGTGTGCCTGTTCACCGACTTCGACAGCC |
| | | AGACCAACGTGTCCCAGAGCAAGGACAGCGACGTGTACATCACCGACA |
| | | AGACAGTGCTGGACATGCGGAGCATGGACTTCAAGAGCAATAGCGCCG |
| | | TGGCCTGGTCCAACAAGAGCGATTTTGCCTGCGCCAACGCCTTCAACA |
| | | ACAGCATCATCCCCGAGGACACATTCTTCCCAAGTCCTGAGAGCAGCTG |
| | | CGACGTGAAGCTGGTGGAAAAGAGCTTCGAGACAGACACCAACCTGA |
| | | ACTTCCAGAACCTGAGCGTGATCGGCTTCAGAATCCTGCTGCTCAAGGT |
| | | GGCCGGCTTTAACCTGCTGATGACCCTGAGACTGTGGTCTAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | (β chain] (SEQ ID No. 35) |
| | | ATGGGCTTCAGACTGCTGTGCTGCGTGGCCTTTTGTCTGCTTGGAGCCG |
| | | GACCTGTGGATAGCGGAGTGACACAGACCCCTAAACACCTGATCACCG |
| | | CCACAGGCCAGCGCGTGACACTGAGATGTTCTCCAAGATCCGGCGACC |
| | | TGTCCGTGTATTGGTATCAGCAGTCTCTGGACCAGGGCCTGCAGTTCCTG |
| | | ATCCAGTACTACAACGGCGAGGAAAGAGCCAAGGGCAACATCCTGGA |
| | | AAGATTCAGCGCCCAGCAGTTCCCCGATCTGCACAGCGAACTGAACCT |
| | | GTCTAGCCTGGAACTGGGCGACAGCGCCCTGTATTTTTGTGCCTCTGGC |
| | | AGCGGCATCCGGCAAGAGACACAGTATTTTGGCCCTGGCACACGGCTG |
| | | CTGGTGCTGGAAGATCTGAAGAACGTGTTCCCACCTGAGGTGGCCGTGT |
| | | TTGAGCCTTCTGAGGCCGAGATCTCTCACACCCAGAAAGCCACACTCGT |
| | | GTGTCTGGCCACCGGCTTCTATCCCGATCACGTGGAACTGTCTTGGTGG |
| | | GTCAACGGCAAAGAGGTGCACAGCGGCGTTAGCACAGACCCTCAGCC |
| | | TCTGAAAGAGCAGCCCGCTCTGAACGACAGCAGATACTGTCTGTCCAG |
| | | CAGGCTGAGAGTGTCCGCCACCTTCTGGCAGAACCCCAGAAACCACTT |
| | | CAGATGCCAGGTCCAGTTCTACGGCCTGAGCGAGAATGACGAGTGGAC |
| | | CCAGGATAGGGCCAAGCCTGTGACTCAGATTGTGTCTGCCGAAGCCTG |
| | | GGGCAGAGCCGATTGTGGCTTTACAAGCGAGAGCTACCAGCAGGGCG |
| | | TGCTGAGCGCCACAATCCTGTATGAGATCCTGCTGGGCAAAGCCACTCT |
| | | GTACGCTGTGCTGGTGTCTGCCCTGGTGCTGATGGCCATGGTCAAGAGA |
| | | AAGGACTCCAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | (eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 16) |
| | | MKTFAGFSFLFLWLQLDCMSRGEDVEQSLFLSVREGDSSVINCTYTDSSST |
| | | YLYWYKQEPGAGLQLLTYIFSNMDMKQDQRLTVLLNKKDKHLSLRIADTQ |
| | | TGDSAIYF [symbol] GKGTTLSVSSNIQNPDPAVYQLRDSKSSDKSVCL |
| | | FTDFDSQTNVSQSKDSDVYITDKTVLDMRSMDFKSNSAVAWSNKSDFA |
| | | CANAFNNSIIPEDTFFPSPESSCDVKLVEKSFETDTNLNFQNLSVIGFRILLL |
| | | KVAGFNLLMTLRLWSS |
| | | [P2A] (SEQ ID No. 53) |
| | | GSGATNFSLLKQAGDVEENPGP |
| | | [β chain] [symbol] (SEQ ID No. 17) |
| | | MGFRLLCCVAFCLLGAGPVDSGVTQTPKHLITATGQRVTLRCSPRSGDLSV |
| | | YWYQQSLDQGLQFLIQYYNGEERAKGNILERFSAQQFPDLHSELNLSSLE |
| | | LGDSALYF [symbol] GPGTRLLVLEDLKNVFPPEVAVFEPSEAEIS |
| | | HTQKATLVCLATGFYPDHVELSWWVNGKEVHSGVSTDPQPLKEQPALND |
| | | SRYCLSSRLRVSATFWQNPRNHERCQVQFYGLSENDEWTQDRAKPVTQI |
| | | VSAEAWGRADCGFTSESYQQGVLSATILYEILLGKATLYAVLVSALVLMAM |
| | | VKRKDSRGLIN |
| | | (T2A) (SEQ ID No. $4) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGQTLVNRIELKGIDFKEDGNILGHKLEYNYNSHNV |
| | | YIMADKQKNGIKVNFKIRHNIEDGSVQLADHYQQNTPIGDGPVLLPDNH |
| | | YLSTQSALSKDPNEKRDHMVLLEFVTAAGITIGMDELYK* |
| EUMR1-14 | Nucleotide | [α chain] (SEQ ID No. 36) |
| | | ATGGAGACCCTCTTGGGCCTGCTTATCCTTTGGCTGCAGCTGCAATGGG |
| | | TGAGCAGCAAACAGGAGGTGACGCAGATTCCTGCAGCTCTGAGTGTCC |
| | | CAGAAGGAGAAAACTTGGTTCTCAACTGCAGTTTCACTGATAGCGCTAT |
| | | TTACAACCTCCAGTGGTTTAGGCAGGACCCTGGGAAAGGTCTCACATCT |
| | | CTGTTGCTTATTCAGTCAAGTCAGAGAGAGCAAACAAGTGGAAGACTT |
| | | AATGCCTCGCTGGATAAATCATCAGGACGTAGTACTTTATACATTGCAGC |
| | | TTCTCAGCCTGGTGACTCAGCCACCTACCTCTGTGCTGTGCCCTCTAACT |
| | | TTGGAAATGAGAAATTAACCTTTGGGACTGGAACAAGACTCACCATCAT |
| | | ACCCAATATCCAGAACCCTGACCCTGCCGTGTACCAGCTGAGAGACTCT |
| | | AAATCCAGTGACAAGTCTGTCTGCCTATTCACCGATTTTGATTCTCAAAC |
| | | AAATGTGTCACAAAGTAAGGATTCTGATGTGTATATCACAGACAAAACTG |
| | | TGCTAGACATGAGGTCTATGGACTTCAAGAGCAACAGTGCTGTGGCCTG |
| | | GAGCAACAAATCTGACTTTGCATGTGCAAACGCCTTCAACAACAGCATT |
| | | ATTCCAGAAGACACCTTCTTCCCCAGCCCAGAAAGTTCCTGTGATGTCA |
| | | AGCTGGTCGAGAAAAGCTTTGAAACAGATACGAACCTAAACTTTCAAA |
| | | ACCTGTCAGTGATTGGGTTCCGAATCCTCCTCCTGAAAGTGGCCGGGTT |
| | | TAATCTGCTCATGACGCTGCGGCTGTGGTCCAGC |
| | | [P2A] (SEQ ID No. 50) |
| | | GGAAGGGGCGCTACGAACTTTAGTCTGCTCAAACAGGCTGGGGATGTA |
| | | GAGGAAAATCCGGGGCCT |
| | | [β chain] (SEQ ID No. 37) |
| | | ATGCTGCTGCTTCTGCTGCTTCTGGGGCCAGGCTCCGGGCTTGGTGCTG |
| | | TCGTCTCTCAACATCCGAGCTGGGTTATCTGTAAGAGTGGAACCTCTGTG |
| | | AAGATCGAGTGCCGTTCCCTGGACTTTCAGGCCACAACTATGTTTTGGT |
| | | ATCGTCAGTTCCCGAAACAGAGTCTCATGCTGATGGCAACTTCCAATGA |
| | | GGGCTCCAAGGCCACATACGAGCAAGGCGTCGAGAAGGACAAGTTTC |
| | | TCATCAACCATGCAAGCCTGACCTTGTCCACTCTGACAGTGACCAGTGC |
| | | CCATCCTGAAGACAGCAGCTTCTACATCTGCAGTGCTAGAGCCCTATATA |
| | | CAGGGGAAGATAGCAATCAGCCCCAGCATTTTGGTGATGGGACTCGAC |
| | | TCTCCATCCTAGAGGACCTGAAGAACGTGTTCCCGCCGGAAGTGGCAGT |
| | | CTTCGAACCTAGCGAGGCAGAAATAAGCCATACTCAGAAAGCGACCCT |
| | | GGTATGCCTGGCCACAGGATTCTATCCCGACCATGTCGAGCTCAGCTGG |
| | | TGGGTAAACGGTAAAGAGGTACATAGTGGTGTGTCAACAGACCCTCAGC |
| | | CCCTTAAAGAGCAACCTGCCCTTAACGATAGTCGGTATTGCTTGTCATCT |
| | | AGGCTCAGAGTTTCCGCCACGTTTTGGCAGAATCCTAGGAACCATTTTC |
| | | GCTGCCAGGTCCAATTCTACGGGCTCTCAGAAAACGACGAATGGACCC |
| | | AGGACAGAGCGAAGCCAGTAACTCAGATAGTCAGCGCTGAAGCATGG |
| | | GGACGAGCGGATTGCGGATTTACGAGCGAGTCCTACCAGCAAGGCGTT |
| | | CTCTCCGCCACCATCCTTTACGAGATTTTGCTCGGGAAGGCCACATTGTA |
| | | CGCCGTATTGGTAAGTGCGCTTGTCCTGATGGCCATGGTCAAGAGAAAA |
| | | GATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 18) |
| | | METLLGLLILWLQLQWVSSKQEVTQIPAALSVPEGENLVLNCSFTDSAIYN |
| | | LQWFRQDPGKGLTSLLLIQSSQREQTSGRLNASLDKSSGRSTLYIAASQPG |
| | | DSATYL [symbol] GTGTRLTHIIPNIQNPDPAVYQLRDSKSSDKSV |
| | | CLFTDFDSQTNVSQSKDSDVYITDKTVLDMRSMDFKSNSAVAWSNKSD |
| | | FACANAFNNSIIPEDTFFPSPESSCDVKLVEKSFETDTNLNFQNLSVIGFRIL |
| | | LLKVAGFNLLMTLRLWSS |
| | | (P2A] (SEQ ID No. 53) |
| | | GSGATNFSLLKQAGDVEENPGP |
| | | [β chain] [symbol] (SEQ ID No. 19) |
| | | MLLLLLLLGPGSGLGAVVSQHPSWVICKSGTSVKIECRSLDFQATTMFWY |
| | | RQFPKQSLMLMATSNEGSKATYEQGVEKDKFLINHASLTLSTLTVTSAMPE |
| | | DSSFY [symbol] GDGTRLSILEDLKNVEPPEVAVFEPSEA |
| | | EISHTQKATLVCLATGFYPDHVELSWWVNGKEVHSGVSTDPQPLKEQPAL |
| | | NDSRYCLSSRLRVSATFWQNPRNHFRCQVQFYGLSENDEWTQDRAKPV |
| | | TQIVSAEAWGRADCGFTSESYQQGVLSATILYEILIGKATLYAVLVSALVLM |
| | | AMVKRKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNH |
| | | YLSTQSALSKQPNEKRDHMVLLEFVTAAGITLGMDELYK* |
| EUMR1-31 | Nucleotide | [α chain] (SEQ ID No. 38) |
| | | ATGGCCTGCCCCGGCTTCCTGTGGGCCCTGGTGATCAGCACCTGCCTGG |
| | | AGTTCAGCATGGCCCAGACCGTGACCCAGAGCCAGCCCGAGATGAGC |
| | | GTGCAGGAGGCCGAGACCGTGACCCTGAGCTGCACCTACGACACCAG |
| | | CGAGAGCGACTACTACCTGTTCTGGTACAAGCAGCCCCCCAGCAGGCA |
| | | GATGATCCTGGTGATCAGGCAGGAGGCCTACAAGCAGCAGAACGCCAC |
| | | CGAGAACAGGTTCAGCGTGAACTTCCAGAAGGCCGCCAAGAGCTTCA |
| | | GCCTGAAGATCAGCGACAGCCAGCTGGGCGACGCCGCCATGTACTTCT |
| | | GCGCCTACAGGAGCGCCGTGAACGCCAGGCTGATGTTCGGCGACGGC |
| | | ACCCAGCTGGTGGTGAAGCCCAACATCCAGAACCCCGACCCCGCCGTG |
| | | TACCAGCTGAGGGACAGCAAGAGCAGCGACAAGAGCGTGTGCCTGTT |
| | | CACCGACTTCGACAGCCAGACCAACGTGAGCCAGAGCAAGGACAGCG |
| | | ACGTGTACATCACCGACAAGACCGTGCTGGACATGAGGAGCATGGACT |
| | | TCAAGAGCAACAGCGCCGTGGCCTGGAGCAACAAGAGCGACTTCGCC |
| | | TGCGCCAACGCCTTCAACAACAGCATCATCCCCGAGGACACCTTCTTCC |
| | | CCAGCCCCGAGAGCAGCTGCGACAAGCTGGTGGAGAAGAGCTTCGAG |
| | | ACCGACACCAACCTGAACTTCCAGAACCTGAGCGTGATCGGCTTCAGA |
| | | ATCCTGCTGCTGAAGGTGGCCGGCTTCAACCTGCTGATGACCCTGAGGC |
| | | TGTGGAGCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [β chain] (SEQ ID No. 39) |
| | | ATGACCATCAGGCTGCTGTGCTACATGGGCTTCTACTTCCTGGGGGCCG |
| | | GCCTGATGGAGGCCGACATCTACCAGACCCCCAGGTACCTGGTGATCG |
| | | GCACCGGCAAGAAGATCACCCTGGAGTGCAGCCAGACCATGGGCCAC |
| | | GACAAGATGTACTGGTACCAGCAGGACCCCGGCATGGAGCTGCACCTG |
| | | ATCCACTACAGCTACGGCGTGAACAGCACCGAGAAGGGCGACCTGAG |
| | | CAGCGAGAGCACCGTGAGCAGGATCAGGACCGAGCACTTCCCCCTGA |
| | | CCCTGGAGAGCGCCAGGCCCAGCCACACCAGCCAGTACCTGTGCGCC |
| | | AGCAGGGGCCTGGCCGAGTTCACCGACACCCAGTACTTCGGCCCCGGC |
| | | ACCAGGCTGACCGTGCTGGACCTGAAGAACGTGTTCCCGCCGGAAGTG |
| | | GCAGTCTTCGAACCTAGCGAGGCAGAAATAAGCCATACTCAGAAAGCG |
| | | ACCCTGGTATGCCTGGCCACAGGATTCTATCCCGACCATGTCGAGCTCA |
| | | GCTGGTGGGTAAACGGTAAAGAGGTACATAGTGGTGTGTCAACAGACC |
| | | CTCAGCCCCTTAAAGAGCAACCTGCCCTTAACGATAGTCGGTATTGCTTG |
| | | TCATCTAGGCTCAGAGTTTCCGCCACGTTTTGGCAGAATCCTAGGAACC |
| | | ATTTTCGCTGCCAGGTCCAATTCTACGGGCTCTCAGAAAACGACGAATG |
| | | GACCCAGGACAGAGCGAAGCCAGTAACTCAGATAGTCAGCGCTGAAG |
| | | CATGGGGACGAGCGGATTGCGGATTTACGAGCGAGTCCTACCAGCAAG |
| | | GCGTTCTCTCCGCCACCATCCTTTACGAGATTTTGCTCGGGAAGGCCAC |
| | | ATTGTACGCCGTATTGGTAAGTGCGCTTGTCCTGATGGCCATGGTCAAGA |
| | | GAAAAGATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 20) |
| | | MACPGFLWALVISTCLEFSMAQTVTQSQPEMSVQEAETVTLSCTYDTSES |
| | | DYYLFWYKQPPSRDMILVIRQEAYKQQNATENRFSVNFQKAAKSFSLKIS |
| | | DSQLGDAAMYF [symbol] GDGTQLVVKPNIQNPDPAVYQLRD |
| | | SKSSDKSVCLFTDFDSQTNVSQSKDSDVYITDKTVLDMRSMDFKSNSAV |
| | | AWSNKSDFACANAFNNSIIPEDTFFPSPESSCDKIVEKSFETDTNINFQNL |
| | | SVIGFRILLLKVAGFNLLMTLRLWSS |
| | | [P2A] (SEQ ID No. 53) |
| | | GSGATNFSLLKQAGDVEENPGP |
| | | [β chain] (SEQ ID No. 21) |
| | | MTIRLLCYMGFYFLGAGLMEADIYQTPRYLVIGTGKKITLECSQTMGHDKM |
| | | YWYQQDPGMELHLIHYSYGVNSTEKGDLSSESTVSRIRTEHFPLTLESARP |
| | | SHTSQYL [symbol] GPGTRLTVLDLKNVEPPEVAVFEPSEAEIS |
| | | HTQKATIVCLATGFYPDHVELSWWVNGKEVHSGVSTDPQPLKEQPALND |
| | | SRYCLSSRLRVSATFWQNPRNHFRCQVQFYGLSENDEWTQDRAKPVTQI |
| | | VSAEAWGRADCGFTSESYQQGVLSATILYEILIGKATLYAVIVSALVLMAM |
| | | VKRKDSRGLIN |
| | | [T2A] (SEQ ID No. 54) |
| | | GSGEGRGSLLTCGDVEENPGP |
| | | [eGFP] (SEQ ID No. S5) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTIVNRIELKGIDFKEDGNILGHKLEYNYNSHNV |
| | | YIMADKQKNGIKVNFKIRHNIEDGSVQLADHYQQNTPIGDGPVLLPDNH |
| | | YLSTQSALSKDPNEKRDHMVLLEFVTAAGITLGMDELYK^{∧} |
| EUMR1-32 | Nucleotide | [α chain] (SEQ ID No. 40) |
| | | ATGGCCTGCCCCGGCTTCCTGTGGGCCCTGGTGATCAGCACCTGCCTGG |
| | | AGTTCAGCATGGCCCAGACCGTGACCCAGAGCCAGCCCGAGATGAGC |
| | | GTGCAGGAGGCCGAGACCGTGACCCTGAGCTGCACCTACGACACCAG |
| | | CGAGAGCGACTACTACCTGTTCTGGTACAAGCAGCCCCCCAGCAGGCA |
| | | GATGATCCTGGTGATCAGGCAGGAGGCCTACAAGCAGCAGAACGCCAC |
| | | CGAGAACAGGTTCAGCGTGAACTTCCAGAAGGCCGCCAAGAGCTTCA |
| | | GCCTGAAGATCAGCGACAGCCAGCTGGGCGACGCCGCCATGTACTTCT |
| | | GCGCCTACAGGAGCGCCCTGAACGCCAGGCTGATGTTCGGCGACGGC |
| | | ACCCAGCTGGTGGTGAAGCCCAACATCCAGAACCCCGACCCCGCCGTG |
| | | TACCAGCTGAGGGACAGCAAGAGCAGCGACAAGAGCGTGTGCCTGTT |
| | | CACCGACTTCGACAGCCAGACCAACGTGAGCCAGAGCAAGGACAGCG |
| | | ACGTGTACATCACCGACAAGACCGTGCTGGACATGAGGAGCATGGACT |
| | | TCAAGAGCAACAGCGCCGTGGCCTGGAGCAACAAGAGCGACTTCGCC |
| | | TGCGCCAACGCCTTCAACAACAGCATCATCCCCGAGGACACCTTCTTCC |
| | | CCAGCCCCGAGAGCAGCTGCGACGTGAAGCTGGTGGAGAAGAGCTTC |
| | | GAGACCGACACCAACCTGAACTTCCAGAACCTGAGCGTGATCGGCTTC |
| | | AGAATCCTGCTGCTGAAGGTGGCCGGCTTCAACCTGCTGATGACCCTGA |
| | | GGCTGTGGAGCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [B chain] (SEQ ID No. 41) |
| | | ATGACCATCAGGCTGCTGTGCTACATGGGCTTCTACTTCCTGGGCGCCG |
| | | GCCTGATGGAGGCCGACATCTACCAGACCCCCAGGTACCTGGTGATCG |
| | | GCACCGGCAAGAAGATCACCCTGGAGTGCAGCCAGACCATGGGCCAC |
| | | GACAAGATGTACTGGTACCAGCAGGACCCCGGCATGGAGCTGCACCTG |
| | | ATCCACTACAGCTACGGCGTGAACAGCACCGAGAAGGGCGACCTGAG |
| | | CAGCGAGAGCACCGTGAGCAGGATCAGGACCGAGCACTTCCCCCTGA |
| | | CCCTGGAGAGCGCCAGGCCCAGCCACACCAGCCAGTACCTGTGCGCC |
| | | AGCAGCGAGGCCAGGGGCCTGGCCGAGTTCACCGACACCCAGTACTT |
| | | CGGCCCCGGCACCAGGCTGACCGTGCTGGAGGACCTGAAGAACGTGT |
| | | TCCCGCCGGAAGTGGCAGTCTTCGAACCTAGCGAGGCAGAAATAAGCC |
| | | ATACTCAGAAAGCGACCCTGGTATGCCTGGCCACAGGATTCTATCCCGA |
| | | CCATGTCGAGCTCAGCTGGTGGGTAAACGGTAAAGAGGTACATAGTGGT |
| | | GTGTCAACAGACCCTCAGCCCCTTAAAGAGCAACCTGCCCTTAACGATA |
| | | GTCGGTATTGCTTGTCATCTAGGCTCAGAGTTTCCGCCACGTTTTGGCAG |
| | | AATCCTAGGAACCATTTTCGCTGCCAGGTCCAATTCTACGGGCTCTCAG |
| | | AAAACGACGAATGGACCCAGGACAGAGCGAAGCCAGTAACTCAGATA |
| | | GTCAGCGCTGAAGCATGGGGACGAGCGGATTGCGGATTTACGAGCGA |
| | | GTCCTACCAGCAAGGCGTTCTCTCCGCCACCATCCTTTACGAGATTTTGC |
| | | TCGGGAAGGCCACATTGTACGCCGTATTGGTAAGTGCGCTTGTCCTGAT |
| | | GGCCATGGTCAAGAGAAAAGATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 22) |
| | | MACPGELWALVISTCLEFSMAQTVTQSQPEMSVQEAETVTLSCTYDTSES |
| | | DYYLFWYKQPPSRQMILVIRQEAYKQQNATENRFSVNFQKAAKSESLKIS |
| | | DSQLGDAAMYF [symbol] GDGTQLVVKPNIQNPDPAVYQLRD |
| | | SKSSDKSVCLFTDFDSQTNVSQSKDSDVYITDKTVLDMRSMDFKSNSAV |
| | | AWSNKSDFACANAFNNSHPEDTFFPSPESSCQVKLVEKSFETDTNLNFQN |
| | | LSVIGFRILLLKVAGFNLLMTLRLWSS |
| | | [P2A] (SEQ ID No. 53) |
| | | GSGATNFSLLKQAGDVEENPGP |
| | | [β chain] [symbol] (SEQ ID No. 23) |
| | | MTIRLLCYMGFYFLGAGLMEADIYQTPRYLVIGTGKKITLECSQTMGHDKM |
| | | YWYQQDPGMELHLIHYSYGVNSTEKGDLSSESTVSRIRTEHFPLTLESARP |
| | | SHTSQYL [symbol] GPGTRLTVLEDLKNVEPPEVAVFEPSE |
| | | AEISHTQKATLVCLATGFYPDHVELSWWVNGKEVHSGVSTDPQPLKEQP |
| | | ALNDSRYCLSSRLRVSATFWQNPRNHERCQVQFYGLSENDEWTQDRAK |
| | | PVTQIVSAEAWGRADCGFTSESYQQGVLSATILYEILLGKATLYAVLVSALVE |
| | | MAMVKRKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNH |
| | | YLSTQSALSKDPNEKRDHMVLLEFVTAAGITLGMDELYK* |
| EUMR1-33 | Nucleotide | [α chain] (SEQ ID No. 42) |
| | | ATGGCCATGCTGCTGGGCGCCAGCGTGCTGATCCTGTGGCTGCAGCCC |
| | | GACTGGGTGAACAGCCAGCAGAAGAACGACGACCAGCAGGTGAAGC |
| | | AGAACAGCCCCAGCCTGAGCGTGCAGGAGGGCAGGATCAGCATCCTG |
| | | AACTGCGACTACACCAACAGCATGTTCGACTACTTCCTGTGGTACAAGA |
| | | AGTACCCCGCCGAGGGCCCCACCTTCCTGATCAGCATCAGCAGCATCAA |
| | | GGACAAGAACGAGGACGGCAGGTTCACCGTGTTCCTGAACAAGAGCG |
| | | CCAAGCACCTGAGCCTGCACATCGTGCCCAGCCAGCCCGGCGACAGC |
| | | GCCGTGTACTTCTGCGCCGCCCAGATCTACAACCAGGGGGGCAAGCTG |
| | | ATCTTCGGCCAGGGCACCGAGCTGAGCGTGAAGCCCAACATCCAGAAC |
| | | CCCGACCCCGCCGTGTACCAGCTGAGGGACAGCAAGAGCAGCGACAA |
| | | GAGCGTGTGCCTGTTCACCGACTTCGACAGCCAGACCAACGTGAGCCA |
| | | GAGCAAGGACAGCGACGTGTACATCACCGACAAGACCGTGCTGGACAT |
| | | GAGGAGCATGGACTTCAAGAGCAACAGCGCCGTGGCCTGGAGCAACA |
| | | AGAGCGACTTCGCCTGCGCCAACGCCTTCAACAACAGCATCATCCCCG |
| | | AGGACACCTTCTTCCCCAGCCCCGAGAGCAGCTGCGACGTGAAGCTGG |
| | | TGGAGAAGAGCTTCGAGACCGACACCAACCTGAACTTCCAGAACCTGA |
| | | GCGTGATCGGCTTCAGAATCCTGCTGCTGAAGGTGGCCGGCTTCAACCT |
| | | GCTGATGACCCTGAGGCTGTGGAGCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [β chain] (SEQ ID No. 43) |
| | | ATGGGCATCAGGCTGCTGTGCAGGGTGGCCTTCTGCTTCCTGGCCGTGG |
| | | GCCTGGTGGACGTGAAGGTGACCCAGAGCAGCAGGTACCTGGTGAAG |
| | | AGGACCGGCGAGAAGGTGTTCCTGGAGTGCGTGCAGGACATGGACCA |
| | | CGAGAACATGTTCTGGTACAGGCAGGACCCCGGCCTGGGCCTGAGGCT |
| | | GATCTACTTCAGCTACGACGTGAAGATGAAGGAGAAGGGCGACATCCC |
| | | CGAGGGCTACAGCGTGAGCAGGGAGAAGAAGGAGAGGTTCAGCCTG |
| | | ATCCTGGAGAGCGCCAGCACCAACCAGACCAGCATGTACCTGTGCGCC |
| | | AGCAGCTTCAGCAGCGGCAAGCAGTACTTCGGCCCCGGCACCAGGCT |
| | | GACCGTGACCGAGGACCTGAAGAACGTGTTCCCCCCCGAGGTGGCCGT |
| | | GTTCGAACCTAGCGAGGCAGAAATAAGCCATACTCAGAAAGCGACCCT |
| | | GGTATGCCTGGCCACAGGATTCTATCCCGACCATGTCGAGCTCAGCTGG |
| | | TGGGTAAACGGTAAAGAGGTACATAGTGGTGTGTCAACAGACCCTCAGC |
| | | CCCTTAAAGAGCAACCTGCCCTTAACGATAGTCGGTATTGCTTGTCATCT |
| | | AGGCTCAGAGTTTCCGCCACGTTTTGGCAGAATCCTAGGAACCATTTTC |
| | | GCTGCCAGGTCCAATTCTACGGGCTCTCAGAAAACGACGAATGGACCC |
| | | AGGACAGAGCGAAGCCAGTAACTCAGATAGTCAGCGCTGAAGCATGG |
| | | GGACGAGCGGATTGCGGATTTACGAGCGAGTCCTACCAGCAAGGCGTT |
| | | CTCTCCGCCACCATCCTTTACGAGATTTTGCTCGGGAAGGCCACATTGTA |
| | | CGCCGTATTGGTAAGTGCGCTTGTCCTGATGGCCATGGTCAAGAGAAAA |
| | | GATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCGGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGGGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 24) |
| | | MAMLLGASVLILWLQPDWVNSQQKNDDQQVKQNSPSLSVQEGRISILN |
| | | CDYTNSMEDYFLWYKKYPAEGPTFLISISSIKDKNEDGRFTVFLNKSAKHLS |
| | | LHIVPSQPGDSAVYF [symbol] TELSVKPNIQNPDPAVYQ |
| | | LRDSKSSDKSVCLFTDFDSQTNVSQSKDSDVYITDKTVLDMRSMDFKSN |
| | | SAVAWSNKSDFACANAFNNSIIPEDTFFPSPESSCDVKLVEKSFETDTNLN |
| | | FQNLSVIGFRILLLKVAGFNLLMTLRLWSS |
| | | [P2A] (SEQ ID No. 53) |
| | | GSGATNFSLLKQAGDVEENPGP |
| | | [ß chain] [symbol] (SEQ ID No. 25) |
| | | MGIRLLCRVAFCFLAVGLVDVKVTQSSRYLVKRTGEKVFLECVQDMDHEN |
| | | MFWYRQDPGLGLRLIYFSYDVKMKEKGDIPEGYSVSREKKERFSLILESAST |
| | | NQTSMYL [symbol] RLTVTEDLKNVFPPEVAVFEPSEAEISHT |
| | | QKATIVCLATGFYPDHVELSWWVNGKEVHSGVSTDPQPLKEQPALNDSR |
| | | YCLSSRLRVSATFWQNPRNHFRCQVQFYGLSENDEWTQDRAKPVTQIVS |
| | | AEAWGRADCGFTSESYQQGVLSATILYEILLGKATLYAVIVSALVLMAMVK |
| | | RKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNH |
| | | YESTQSALSKDPNEKRDHMVLLEFVTAAGITLGMDELYK* |
| EUMR1-36 | Nucleotide | [α chain] (SEQ ID No. 44) |
| | | ATGAAGACATTTGCTGGATTTTCGTTCCTGTTTTTGTGGCTGCAGCTGGA |
| | | CTGTATGAGTAGAGGAGAGGATGTGGAGCAGAGTCTTTTCCTGAGTGTC |
| | | CGAGAGGGAGACAGCTCCGTTATAAACTGCACTTACACAGACAGCTCC |
| | | TCCACCTACTTATACTGGTATAAGCAAGAACCTGGAGCAGGTCTCCAGTT |
| | | GCTGACGTATATTTTTTCAAATATGGACATGAAACAAGACCAAAGACTC |
| | | ACTGTTCTATTGAATAAAAAGGATAAACATCTGTCTCTGCGCATTGCAGA |
| | | CACCCAGACTGGGGACTCAGCTATCTACTTCTGTGCAGAGATCAAAAGA |
| | | ACTGCTCTGATCTTTGGGAAGGGAACCACCTTATCAGTGAGTTCCAATAT |
| | | CCAGAACCCTGACCCTGCCGTGTACCAGCTGAGAGACTCTAAATCCAGT |
| | | GACAAGTCTGTCTGCCTATTCACCGATTTTGATTCTCAAACAAATGTGTC |
| | | ACAAAGTAAGGATTCTGATGTGTATATCACAGACAAAACTGTGCTAGAC |
| | | ATGAGGTCTATGGACTTCAAGAGCAACAGTGCTGTGGCCTGGAGCAAC |
| | | AAATCTGACTTTGCATGTGCAAACGCCTTCAACAACAGCATTATTCCAG |
| | | AAGACACCTTCTTCCCCAGCCCAGAAAGTTCCTGTGATGTCAAGCTGGT |
| | | CGAGAAAAGCTTTGAAACAGATACGAACCTAAACTTTCAAAACCTGTC |
| | | AGTGATTGGGTTCCGAATCCTCCTCCTGAAAGTGGCCGGGTTTAATCTGC |
| | | TCATGACGCTGGGGCTGTGGTCCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [ß chain] (SEQ ID No. 45) |
| | | ATGGGCTTCAGGCTCCTCTGCTGTGTGGCCTTTTGTCTCCTGGGAGCAG |
| | | GCCCAGTGGATTCTGGAGTCACACAAACCCCAAAGCACCTGATCACAG |
| | | CAACTGGACAGCGAGTGACGCTGAGATGCTCCCCTAGGTCTGGAGACC |
| | | TCTCTGTGTACTGGTACCAACAGAGCCTGGACCAGGGCCTCCAGTTCCT |
| | | CATTCAGTATTATAATGGAGAAGAGAGAGCAAAAGGAAACATTCTTGAA |
| | | CGATTCTCCGCACAACAGTTCCCTGACTTGCACTCTGAACTAAACCTGA |
| | | GCTCTCTGGAGCTGGGGGACTCAGCTTTGTATTTCTGTGCCAGCAGCGG |
| | | CTCAGGGGAGCAAGAGACCCAGTACTTCGGGCCAGGCACGCGGCTCC |
| | | TGGTGCTCGAGGACCTGAAAAACGTGTTCCCACCCGAGGTCGCTGTGTT |
| | | CGAACCTAGCGAGGCAGAAATAAGCCATACTCAGAAAGCGACCCTGGT |
| | | ATGCCTGGCCACAGGATTCTATCCCGACCATGTCGAGCTCAGCTGGTGG |
| | | GTAAACGGTAAAGAGGTACATAGTGGTGTGTCAACAGACCCTCAGCCCC |
| | | TTAAAGAGCAACCTGCCCTTAACGATAGTCGGTATTGCTTGTCATCTAGG |
| | | CTCAGAGTTTCCGCCACGTTTTGGCAGAATCCTAGGAACCATTTTCGCT |
| | | GCCAGGTCCAATTCTACGGGCTCTCAGAAAACGACGAATGGACCCAGG |
| | | ACAGAGCGAAGCCAGTAACTCAGATAGTCAGCGCTGAAGCATGGGGA |
| | | CGAGCGGATTGCGGATTTACGAGCGAGTCCTACCAGCAAGGCGTTCTC |
| | | TCCGCCACCATCCTTTACGAGATTTTGCTCGGGAAGGCCACATTGTACG |
| | | CCGTATTGGTAAGTGCGCTTGTCCTGATGGCCATGGTCAAGAGAAAAGA |
| | | TTCTAGAGGCTTAATTAAT |
| | | [T2A] (SEQ ID No. S1) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 26) |
| | | MKTFAGFSFLFLWLQLDCMSRGEDVEQSLFLSVREGQSSVINCTYTDSSST |
| | | YLYWYKQEPGAGLQLLTYIFSNMDMKQDQRLTVLLNKKDKHLSLRIADTQ |
| | | TGDSAIYF [symbol] GKGTTLSVSSNIQNPDPAVYQLRDSKSSDKSVCL |
| | | FTDFDSQTNVSQSKDSDVYITDKTVLDMRSMDFKSNSAVAWSNKSDFA |
| | | CANAFNNSIPEDTFFPSPESSCDVKLVEKSFETDTNINFQNLSVIGFRILLL |
| | | KVAGFNLLMTLRLWSS |
| | | (P2A] (SEQ ID No. 53) |
| | | GSGATNFSLLKQAGDVEENPGP |
| | | [β chain] [symbol] (SEQ ID No. 27) |
| | | MGFRLLCCVAFCLIGAGPVDSGVTQTPKHUTATGQRVTLRCSPRSGDLSV |
| | | YWYQQSLDQGLQFLIQYYNGEERAKGNILERFSAQQFPDLHSELNUSSLE |
| | | LGDSALYF [symbol] GPGTRLLVLEDLKNVFPPEVAVFEPSEAEI |
| | | SHTQKATLVCLATGFYPDHVELSWWVNGKEVHSGVSTDPQPLKEQPALN |
| | | DSRYCLSSRLRVSATFWQNPRNHFRCQVQFYGLSENDEWTQDRAKPVT |
| | | QIVSAEAWGRADCGFTSESYQQGVLSATILYEILLGKATLYAVIVSALVLMA |
| | | MVKRKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGQVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVILPDNH |
| | | YESTQSALSKDPNEKRDHMVLLEFVTAAGITLGMDELYK* |
| EUMR1-37 | Nucleotide | [α chain] (SEQ ID No. 46) |
| | | ATGAAGACATTTGCTGGATTTTCGTTCCTGTTTTTGTGGCTGCAGCTGGA |
| | | CTGTATGAGTAGAGGAGAGGATGTGGAGCAGAGTCTTTTCCTGAGTGTC |
| | | CGAGAGGGAGACAGCTCCGTTATAAACTGCACTTACACAGACAGCTCC |
| | | TCCACCTACTTATACTGGTATAAGCAAGAACCTGGAGCAGGTCTCCAGTT |
| | | GCTGACGTATATTTTTTCAAATATGGACATGAAACAAGACCAAAGACTC |
| | | ACTGTTCTATTGAATAAAAAGGATAAACATCTGTCTCTGCGCATTGCAGA |
| | | CACCCAGACTGGGGACTCAGCTATCTACTTCTGTGCAGAGATCAAAAGA |
| | | ACTGCTCTGATCTTTGGGAAGGGAACCACCTTATCAGTGAGTTCCAATAT |
| | | CCAGAACCCTGACCCTGCCGTGTACCAGCTGAGAGACTCTAAATCCAGT |
| | | GACAAGTCTGTCTGCCTATTCACCGATTTTGATTCTCAAACAAATGTGTC |
| | | ACAAAGTAAGGATTCTGATGTGTATATCACAGACAAAACTGTGCTAGAC |
| | | ATGAGGTCTATGGACTTCAAGAGCAACAGTGCTGTGGCCTGGAGCAAC |
| | | AAATCTGACTTTGCATGTGCAAACGCCTTCAACAACAGCATTATTCCAG |
| | | AAGACACCTTCTTCCCCAGCCCAGAAAGTTCCTGTGATGTCAAGCTGGT |
| | | CGAGAAAAGCTTTGAAACAGATACGAACCTAAACTTTCAAAACCTGTC |
| | | AGTGATTGGGTTCCGAATCCTCCTCCTGAAAGTGGCCGGGTTTAATCTGC |
| | | TCATGACGCTGCGGCTGTGGTCCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [ß chain] (SEQ ID No. 47) |
| | | ATGAGCCTCGGGCTCCTGTGCTGTGGGGCCTTTTCTCTCCTGTGGGCAG |
| | | GTCCAGTGAATGCTGGTGTCACTCAGACCCCAAAATTCCGGGTCCTGAA |
| | | GACAGGACAGAGCATGACACTGCTGTGTGCCCAGGATATGAACCATGA |
| | | ATACATGTACTGGTATCGACAAGACCCAGGCATGGGGCTGAGGCTGATT |
| | | CATTACTCAGTTGGTGAGGGTACAACTGCCAAAGGAGAGGTCCCTGATG |
| | | GCTACAATGTCTCCAGATTAAAAAAACAGAATTTCCTGCTGGGGTTGGA |
| | | GTCGGCTGCTCCCTCCCAAACATCTGTGTACTTCTGTGCCAGCAGTTCCG |
| | | GCTCTAGGGGGGTGTACCAAGAGACCCAGTACTTCGGGCCAGGCACGC |
| | | GGCTCCTGGTGCTCGAGGACCTGAAAAACGTGTTCCCACCCGAGGTCG |
| | | CTGTGTTCGAACCTAGCGAGGCAGAAATAAGCCATACTCAGAAAGCGA |
| | | CCCTGGTATGCCTGGCCACAGGATTCTATCCCGACCATGTCGAGCTCAG |
| | | CTGGTGGGTAAACGGTAAAGAGGTACATAGTGGTGTGTCAACAGACCCT |
| | | CAGCCCCTTAAAGAGCAACCTGCCCTTAACGATAGTCGGTATTGCTTGTC |
| | | ATCTAGGCTCAGAGTTTCCGCCACGTTTTGGCAGAATCCTAGGAACCAT |
| | | TTTCGCTGCCAGGTCCAATTCTACGGGCTCTCAGAAAACGACGAATGGA |
| | | CCCAGGACAGAGCGAAGCCAGTAACTCAGATAGTCAGCGCTGAAGCAT |
| | | GGGGACGAGCGGATTGCGGATTTACGAGCGAGTCCTACCAGCAAGGC |
| | | GTTCTCTCCGCCACCATCCTTTACGAGATTTTGCTCGGGAAGGCCACATT |
| | | GTACGCCGTATTGGTAAGTGCGCTTGTCCTGATGGCCATGGTCAAGAGA |
| | | AAAGATTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGGGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 28) |
| | | MKTFAGFSFLFLWLQLDCMSRGEDVEQSLFLSVREGDSSVINCTYTQSSST |
| | | YLYWYKQEPGAGLQLLTYIFSNMDMKQDQRLTVLLNKKDKHLSLRIADTQ |
| | | TGDSAIYF [symbol] GKGTTLSVSSNIQNPDPAVYQLRDSKSSDKSVCL |
| | | FTDFDSQTNVSQSKDSDVYITDKTVLDMRSMDFKSNSAVAWSNKSDFA |
| | | CANAFNNSIIPEDTFFPSPESSCDVKLVEKSFETDTNINFQNLSVIGFRILLI |
| | | KVAGFNLLMTLRLWSS |
| | | [P2A] (SEQ ID No. 53) |
| | | GSGATNFSLLKQAGDVEENPGP |
| | | [β chain] [symbol] (SEQ ID No. 29) |
| | | MSLGLLCCGAFSLLWAGPVNAGVTQTPKFRVLKTGQSMTLLCAQDMNH |
| | | EYMYWYRQDPGMGLRLIHYSVGEGTTAKGEVPDGYNVSRLKKQNFLLGL |
| | | ESAAPSQTSVYF [symbol] GPGTRLLVLEDLKNVEPPEVAV |
| | | FEPSEAFISHTQKATLVCLATGFYPDHVELSWWVNGKEVHSGVSTDPQPL |
| | | KEQPALNDSRYCLSSRLRVSATFWQNPRNHFRCQVQFYGLSENDEWTQ |
| | | DRAKPVTQIVSALAWGRADCGFTSESYQQGVLSATILYBILLGKATLYAVIV |
| | | SALVLMAMVKRKDSRGLIN |
| | | [T2A] (SEQ ID No. S4) |
| | | GSGEGRGSLLTCGDVEENPGP |
| | | [eGFP] (SEQ ID No. SS) |
| | | MVSKGEELFTGVVPILVELDGDVNGHKFSVSGEGEGDATYGKLTLKFCTT |
| | | GKLPVPWPTIVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNH |
| | | YESTQSALSKDPNEKRDHMVLLERVTAAGITIGMDELYK* |
| EUMR1-38 | Nucleotide | [α chain] (SEQ ID No. 48) |
| | | ATGAAGACATTTGCTGGATTTTCGTTCCTGTTTTTGTGGCTGCAGCTGGA |
| | | CTGTATGAGTAGAGGAGAGGATGTGGAGCAGAGTCTTTTCCTGAGTGTC |
| | | CGAGAGGGAGACAGCTCCGTTATAAACTGCACTTACACAGACAGCTCC |
| | | TCCACCTACTTATACTGGTATAAGCAAGAACCTGGAGCAGGTCTCCAGTT |
| | | GCTGACGTATATTTTTTCAAATATGGACATGAAACAAGACCAAAGACTC |
| | | ACTGTTCTATTGAATAAAAAGGATAAACATCTGTCTCTGCGCATTGCAGA |
| | | CACCCAGACTGGGGACTCAGCTATCTACTTCTGTGCAGAACTGGCAGG |
| | | AACTGCTCTGATCTTTGGGAAGGGAACCACCTTATCAGTGAGTTCCAAT |
| | | ATCCAGAACCCTGACCCTGCCGTGTACCAGCTGAGAGACTCTAAATCCA |
| | | GTGACAAGTCTGTCTGCCTATTCACCGATTTTGATTCTCAAACAAATGTGT |
| | | CACAAAGTAAGGATTCTGATGTGTATATCACAGACAAAACTGTGCTAGA |
| | | CATGAGGTCTATGGACTTCAAGAGCAACAGTGCTGTGGCCTGGAGCAA |
| | | CAAATCTGACTTTGCATGTGCAAACGCCTTCAACAACAGCATTATTCCA |
| | | GAAGACACCTTCTTCCCCAGCCCAGAAAGTTCCTGTGATGTCAAGCTGG |
| | | TCGAGAAAAGCTTTGAAACAGATACGAACCTAAACTTTCAAAACCTGTC |
| | | AGTGATTGGGTTCCGAATCCTCCTCCTGAAAGTGGCCGGGTTTAATCTGC |
| | | TCATGACGCTGCGGCTGTGGTCCAGC |
| | | [P2A] (SEQ ID No. 50) |
| | | GGAAGCGGCGCTACGAACTTTAGTCTGCTCAAACAGGCTGGGGATGTA |
| | | GAGGAAAATCCGGGGCCT |
| | | [B chain] (SEQ ID No. 49) |
| | | ATGGGCTTCAGGCTCCTCTGCTGTGTGGCCTTTTGTCTCCTGGGAGCAG |
| | | GCCCAGTGGATTCTGGAGTCACACAAACCCCAAAGCACCTGATCACAG |
| | | CAACTGGACAGCGAGTGACGCTGAGATGCTCCCCTAGGTCTGGAGACC |
| | | TCTCTGTGTACTGGTACCAACAGAGCCTGGACCAGGGCCTCCAGTTCCT |
| | | CATTCAGTATTATAATGGAGAAGAGAGAGCAAAAGGAAACATTCTTGAA |
| | | CGATTCTCCGCACAACAGTTCCCTGACTTGCACTCTGAACTAAACCTGA |
| | | GCTCTCTGGAGCTGGGGGACTCAGCTTTGTATTTCTGTGCCAGCGGGTC |
| | | AGGGATTCGGCAAGAGACCCAGTACTTCGGGCCAGGCACGCGGCTCC |
| | | TGGTGCTCGAGGACCTGAAAAACGTGTTCCCACCCGAGGTCGCTGTGTT |
| | | CGAACCTAGCGAGGCAGAAATAAGCCATACTCAGAAAGCGACCCTGGT |
| | | ATGCCTGGCCACAGGATTCTATCCCGACCATGTCGAGCTCAGCTGGTGG |
| | | GTAAACGGTAAAGAGGTACATAGTGGTGTGTCAACAGACCCTCAGCCCC |
| | | TTAAAGAGCAACCTGCCCTTAACGATAGTCGGTATTGCTTGTCATCTAGG |
| | | CTCAGAGTTTCCGCCACGTTTTGGCAGAATCCTAGGAACCATTTTCGCT |
| | | GCCAGGTCCAATTCTACGGGCTCTCAGAAAACGACGAATGGACCCAGG |
| | | ACAGAGCGAAGCCAGTAACTCAGATAGTCAGCGCTGAAGCATGGGGA |
| | | CGAGCGGATTGCGGATTTACGAGCGAGTCCTACCAGCAAGGCGTTCTC |
| | | TCCGCCACCATCCTTTACGAGATTTTGCTCGGGAAGGCCACATTGTACG |
| | | CCGTATTGGTAAGTGCGCTTGTCCTGATGGCCATGGTCAAGAGAAAAGA |
| | | TTCTAGAGGCTTAATTAAT |
| | | [T2A] (SEQ ID No. 51) |
| | | GGCTCCGGCGAGGGCAGGGGAAGTCTTCTAACATGGGGGGACGTGGA |
| | | GGAAAATCCCGGCCCA |
| | | [eGFP] (SEQ ID No. 52) |
| | | ATGGTGAGCAAGGGCGAGGAGCTGTTCACCGGGGTGGTGCCCATCCTG |
| | | GTCGAGCTGGACGGCGACGTAAACGGCCACAAGTTCAGCGTGTCCGGC |
| | | GAGGGCGAGGGCGATGCCACCTACGGCAAGCTGACCCTGAAGTTCATC |
| | | TGCACCACCGGCAAGCTGCCCGTGCCCTGGCCCACCCTCGTGACCACC |
| | | CTGACCTACGGCGTGCAGTGCTTCAGCCGCTACCCCGACCACATGAAG |
| | | CAGCACGACTTCTTCAAGTCCGCCATGCCCGAAGGCTACGTCCAGGAG |
| | | CGCACCATCTTCTTCAAGGACGACGGCAACTACAAGACCCGCGCCGAG |
| | | GTGAAGTTCGAGGGCGACACCCTGGTGAACCGCATCGAGCTGAAGGG |
| | | CATCGACTTCAAGGAGGACGGCAACATCCTGGGGCACAAGCTGGAGT |
| | | ACAACTACAACAGCCACAACGTCTATATCATGGCCGACAAGCAGAAGA |
| | | ACGGCATCAAGGTGAACTTCAAGATCCGCCACAACATCGAGGACGGCA |
| | | GCGTGCAGCTCGCCGACCACTACCAGCAGAACACCCCCATCGGCGACG |
| | | GCCCCGTGCTGCTGCCCGACAACCACTACCTGAGCACCCAGTCCGCCCT |
| | | GAGCAAAGACCCCAACGAGAAGCGCGATCACATGGTCCTGCTGGAGTT |
| | | CGTGACCGCCGCCGGGATCACTCTCGGCATGGACGAGCTGTACAAGTA |
| | | A |
| | Amino acid | [α chain] [symbol] (SEQ ID No. 30) |
| | | MKTFAGFSFLFLWLQLDCMSRGEDVEQSLFLSVREGDSSVINCTYTDSSST |
| | | YLYWYKQEPGAGLQLLTYIFSNMDMKQDQRLTVLLNKKDKHLSLRIADTQ |
| | | TGDSAIYF [symbol] GKGTTLSVSSNIQNPDPAVYQLRDSKSSDKSVC |
| | | LFTDFDSQINVSQSKDSDVYITDKTVLDMRSMDFKSNSAVAWSNKSDF |
| | | ACANAFNNSIIPEDTFFPSPESSCDVKLVEKSFETDTNLNFQNLSVIGFRILL |
| | | LKVAGENLLMTLRLWSS |
| | | [P2A] (SEQ ID No. 53) |
| | | GSGATNFSLLKQAGDVEENPGP |
| | | [β chain] [symbol] (SEQ ID No. 31) |
| | | MGFRLLCCVAFCLLGAGPVDSGVTQTPKHLITATGQRVTLRCSPRSGDLSV |
| | | YWYQQSLDQGLQFLIQYYNGEERAKGNILERFSAQQFPDLHSELNLSSLE |
| | | LGDSALYF [symbol] GPGTRLLVLEDLKNVEPPEVAVFEPSEAEIS |
| | | HTQKATLVCLATGFYPDHVELSWWVNGKEVHSGVSTDPQPLKEQPALND |
| | | SRYCLSSRLRVSATFWQNPRNHERCQVQFYGLSENDEWTQDRAKPVTQI |
| | | VSAEAWGRADCGFTSESYQQGVLSATILVEILIGKATLYAVIVSALVLMAM |
| | | VKRKDSRGLIN |
| | | [T2A] (SEQ ID No. 54) |
| | | GSGEGRGSLLTCGDVEENPGP |
| | | [eGFP] (SEQ ID No. 55) |
| | | MVSKGEELFTGVVPILVELDGDVNGHKFSVSGEGEGDATYGKLTLKFICTT |
| | | GKLPVPWPTLVTTLTYGVQCFSRYPDHMKQHDFFKSAMPEGYVQERTIFF |
| | | KDDGNYKTRAEVKFEGDTLVNRIELKGIDFKEDGNILGHKLEYNYNSHNV |
| | | YIMADKQKNGIKVNFKIRHNIEDGSVQLADHYQQNTPIGDGPVLLPDNH |
| | | YESTQSALSKDPNEKRDHMVLLEFVTAAGITLGMDELYK* |

3.3 Cloning Using Competent Cell Stbl III

A vector was linearized using restriction enzymes Bam HI and BstB I inside a structure of a vector backbone pELPS3-TRBC-P2A-eGFP. The synthesized MR1 TCR was ligated with the vector after treatment with the same restriction enzyme as an insert.

3.4 Cloning Results

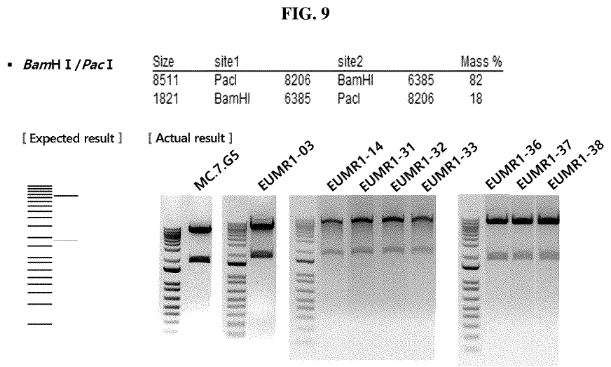

Appropriate enzymes were selected for structural analysis of the cloned lentiviral transfection plasmid, and the results are summarized in FIG. 9. In addition, sequencing was performed on the plasmid [Macrogen, Order No. HC00246947, HC00256796, HC00257025]. As a result of sequencing, it was confirmed that there was no problem in a nucleotide sequence of the constructed plasmid.

### Example 4. Construction of MR1 TCR-Jurkat-NFAT-Luc and Potency Test

4.1 Lentivirus Production

The cloned lentivirus transfection plasmid and three lentivirus packaging plasmids were transfected into Lenti-X 293 cells with a Lipofectamine 3000 transfection agent to produce lentivirus.

4.2 Transduction

A 10% FBS RPMI culture medium was diluted with Protamine sulfate (10 mg/mL) to be a concentration of 10 μg/mL and prepared. The cell number of Jurkat-NFAT-Luciferase was measured. Resuspension was performed with a diluted protamine sulfate culture medium according to a cell concentration of 2×10⁶ cells/mL. 1.5 mL of the cell mixture was added to each well in a 6-well plate. 500 μL of the produced virus was added. The virus was centrifuged under conditions of 25° C., 1200 g, and 2 hours (Spinoculation). After centrifugation, 1.5 mL of the culture medium was added per well and cultured in an incubator at 37° C. and 5% CO₂.

4.3 FACS Analysis for Expression Confirmation

To confirm TCR expression in Jurkat-NFAT-Luciferase, FACS analysis was performed using FACSCelesta to confirm the expression of GFP, a tagged protein.

4.4 Potency Test (Luciferase Based Assay)

The cell number was measured by harvesting effector T cells (MR1 TCR-Jurkat-NFAT-Luc). The cells were centrifuged at 1500 rpm for 5 minutes. After removal of supernatant, the culture medium was added so that the cell concentration was 4.0×10⁵ cells/mL and resuspended.

Target cells (MR1 overexpressed A375) were harvested and the cell number was measured. The cells were centrifuged under conditions of 1500 rpm for 5 minutes. After removal of supernatant, the culture medium was added so that the cell concentration was 4.0×10⁶ cells/mL.

Target cells were serially diluted 3-fold according to an E:T ratio (duplicate). μL/well (effector (E):target (T) ratio=1:10) of target cells was added to a 96-well White Flat bottom Assay Plate, and serially diluted 3-fold up to E:T ratio=1:0.3 using a multipipette. 50 μL of an effector T cell diluted solution was added per well (2.0×10⁴ cells/well). 50 μL of the culture medium was added to wells of a negative control group containing only effector T cells.

The cells were co-cultured in a 37° C., 5% CO₂ incubator for 4 hours. After culturing for 4 hours, a Bright-Glo™ Luciferase Assay reagent was added and reacted for 5 minutes, and then luminescence was measured using a luminometer.

A Fold value of the luminescence values was calculated by using a non-activated group as a negative control by adding the culture medium instead of the target cells.

$$Luminescence Fold value = \frac{Luminescence of co ‐ cultured cell with target cell}{Luminescence of Unactivated cell}$$

4.5 Results of Confirming TCR Expression in Jurkat-NFAT-Luciferase

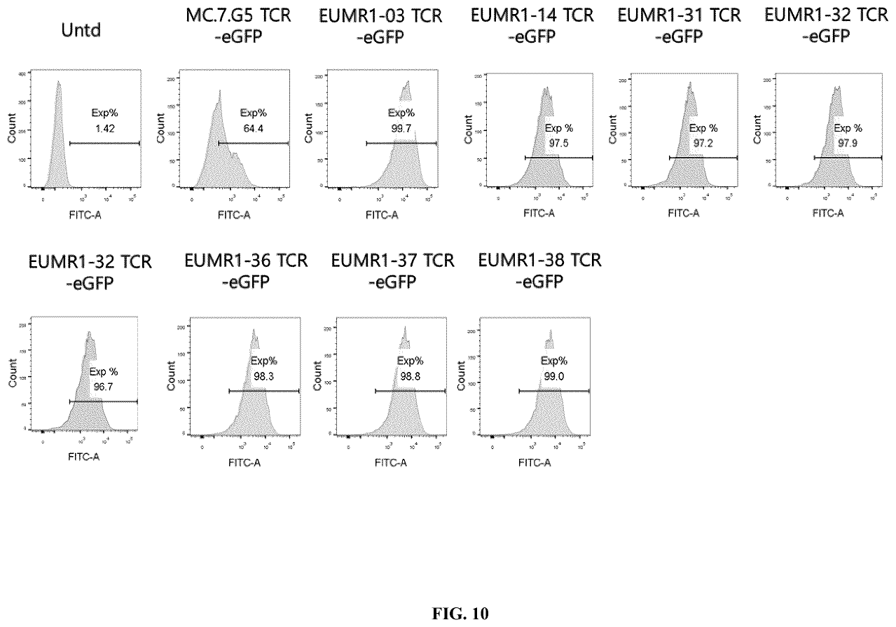

Viral TCR expression was confirmed in Jurkat for all clones. However, since the same virus volume was treated, actual MOI was varied for each clone (FIG. 10).

6.6 Potency Assay Results

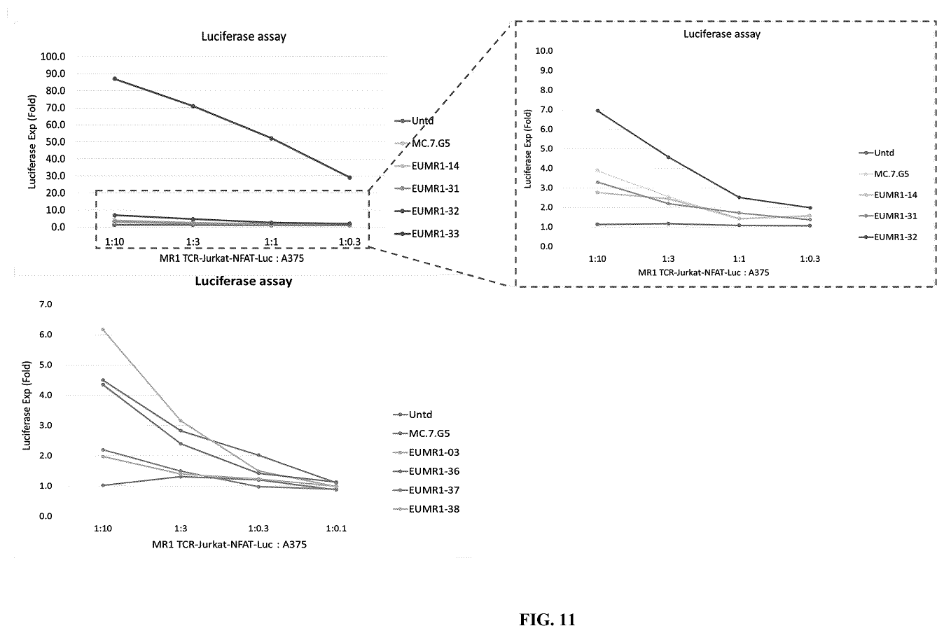

Luciferase-based functional assay was performed using the produced MR1 TCR-Jurkat-NFAT-Luciferase as an effector cell and using an A375-MR1 cell line as a target cell. As a result, when compared to an MC.7.G5 group as a reference group, EUMR1-03, 14, 31, 32, 36, 37, and 38 all showed a similar level of reactivity, and EUMR1-33 clones had a significantly higher activity (FIG. 11).

As described above, specific parts of the present invention have been described in detail, and it will be apparent to those skilled in the art that these specific techniques are merely preferred embodiments, and the scope of the present invention is not limited thereto. Therefore, the substantial scope of the present disclosure will be defined by the appended claims and their equivalents.

### INDUSTRIAL APPLICABILITY

The present invention can be applied as a T-cell therapeutic agent expressing a T-cell receptor applicable to all cancer types regardless of a HLA type, unlike existing customized anti-cancer immune T cell therapeutic agents which are used limitedly according to the expression of cancer antigens depending on a cancer type and a HLA type. These MR1 T cells have the ability to selectively attack only cancer cells without attacking normal cells, thereby increasing an anticancer effect without side effects and exhibiting a synergy even in combination therapy with various existing therapeutic agents.

1. A T-cell receptor binding to MHC class I related protein (MR1) comprising CDR3α selected from the group consisting of SEQ ID NOs: 3, 5, 8, 9, and 13; and CDR3β selected from the group consisting of SEQ ID NOs: 2, 4, 6, 7, 10, 11, and 12.

2. The T-cell receptor of claim 1, wherein the T-cell receptor includes:
CDR3α of SEQ ID NO: 3 and CDR3β of SEQ ID NO: 4;
CDR3α of SEQ ID NO: 5 and CDR3β of SEQ ID NO: 6;
CDR3α of SEQ ID NO: 1 and CDR3β of SEQ ID NO: 7;
CDR3α of SEQ ID NO: 8 and CDR3β of SEQ ID NO: 2;
CDR3α of SEQ ID NO: 9 and CDR3β of SEQ ID NO: 10;
CDR3α of SEQ ID NO: 3 and CDR3β of SEQ ID NO: 11;
CDR3α of SEQ ID NO: 3 and CDR3β of SEQ ID NO: 12; or
CDR3α of SEQ ID NO: 13 and CDR3β of SEQ ID NO: 4.

3. The T-cell receptor of claim 1, comprising an α chain selected from the group consisting of SEQ ID NOs: 14, 16, 18, 20, 22, 24, 26, 28, and 30.

4. The T-cell receptor of claim 1, comprising a β chain selected from the group consisting of SEQ ID NOs: 15, 17, 19, 21, 23, 25, 27, 29, and 31.

5. A nucleic acid encoding the T-cell receptor according to claim 1.

6. The nucleic acid of claim 5, wherein the nucleic acid comprises a sequence selected from the group consisting of SEQ ID NOs: 32 to 49.

7. A vector in which the nucleic acid of claim 5 is cloned.

8. A T cell expressing the T-cell receptor according to claim 1.

9. The T cell of claim 8, the T cell is CD8+.

10. (canceled)

11. A method for treating a tumor or cancer including administering to a subject the T-cell receptor according to any one of claims 1 to 4, the nucleic acid of claim 5, the vector of claim 7, or the T cell of claim 8.


SEQUENCE LISTING (55 sequences)

SEQ ID NO: 1 - protein, 13 aa, Artificial Sequence
  CDR3
CAYRSAVNARLMF

SEQ ID NO: 2 - protein, 18 aa, Artificial Sequence
  CDR3
CASSEARGLAEFTDTQYF

SEQ ID NO: 3 - protein, 11 aa, Artificial Sequence
  CDR3
CAEIKRTALIF

SEQ ID NO: 4 - protein, 14 aa, Artificial Sequence
  CDR3
CASGSGIRQETQYF

SEQ ID NO: 5 - protein, 14 aa, Artificial Sequence
  CDR3
CAVPSNFGNEKLTF

SEQ ID NO: 6 - protein, 18 aa, Artificial Sequence
  CDR3
CSARALYTGEDSNQPQHF

SEQ ID NO: 7 - protein, 15 aa, Artificial Sequence
  CDR3
CASRGLAEFTDTQYF

SEQ ID NO: 8 - protein, 13 aa, Artificial Sequence
  CDR3
CAYRSALNARLMF

SEQ ID NO: 9 - protein, 17 aa, Artificial Sequence
  CDR3
CAAQIYNQGGKLIFGQG

SEQ ID NO: 10 - protein, 16 aa, Artificial Sequence
  CDR3
CASSFSSGKQYFGPGT

SEQ ID NO: 11 - protein, 14 aa, Artificial Sequence
  CDR3
CASSGSGEQETQYF

SEQ ID NO: 12 - protein, 17 aa, Artificial Sequence
  CDR3
CASSSGSSGVYQETQYF

SEQ ID NO: 13 - protein, 11 aa, Artificial Sequence
  CDR3
CAELAGTALIF

SEQ ID NO: 14 - protein, 275 aa, Artificial Sequence
  alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 318 aa, Artificial Sequence
  beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 16 - protein, 271 aa, Artificial Sequence
  alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEIKRTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 314 aa, Artificial Sequence
  beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASGSGIRQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 18 - protein, 273 aa, Artificial Sequence
  alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVPSNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 316 aa, Artificial Sequence
  beta chain
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARALYTGEDSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 20 - protein, 274 aa, Artificial Sequence
  alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDKLVEKSFETDTNLNFQNLSVIGFRILLLKVAGFNLLMTLRLWSS

SEQ ID NO: 21 - protein, 314 aa, Artificial Sequence
  beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASRGLAEFTDTQYFGPGTRLTVLDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 22 - protein, 275 aa, Artificial Sequence
  alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSALNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 318 aa, Artificial Sequence; identical to SEQ ID NO: 15
  beta chain

SEQ ID NO: 24 - protein, 280 aa, Artificial Sequence
  alpha chain
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAQIYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 312 aa, Artificial Sequence
  beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFSSGKQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 26 - protein, 271 aa, Artificial Sequence; identical to SEQ ID NO: 16
  alpha chain

SEQ ID NO: 27 - protein, 314 aa, Artificial Sequence
  beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGSGEQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 28 - protein, 271 aa, Artificial Sequence; identical to SEQ ID NO: 16
  alpha chain

SEQ ID NO: 29 - protein, 317 aa, Artificial Sequence
  beta chain
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSSGSSGVYQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLIN

SEQ ID NO: 30 - protein, 271 aa, Artificial Sequence
  alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAELAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 314 aa, Artificial Sequence; identical to SEQ ID NO: 17
  beta chain

SEQ ID NO: 32 - DNA, 825 bp, Artificial Sequence
  alpha chain
atggcttgtccaggttttttgtgggcgctggtcatctccacttgcctcgaatttagtatggcccagacggtgacgcagtcacagccggagatgtccgttcaagaagctgaaactgtcacacttagttgtacgtatgacacctcagaaagcgactattacttgttttggtataaacaacctccctctcgccagatgatccttgttatacgacaagaggcatacaaacagcagaatgcaactgagaatcggtttagcgtaaactttcaaaaggcggccaaaagtttctctctgaaaatttctgactctcaattgggagacgccgcgatgtatttctgtgcgtataggagcgccgtaaacgctcgacttatgtttggcgacggaactcaactcgtagtcaagcccaacatacaaaatcctgatccggccgtctaccaactgagggattcaaagtcttccgacaagtctgtttgtctgtttacggatttcgacagccaaacgaacgtatcacagtccaaggactccgatgtatatatcaccgataagacagtattggatatgaggtccatggatttcaaaagcaattccgctgtggcctggtcaaataagagcgacttcgcctgtgcgaatgcattcaataattctataatcccagaggacacgttttttcccagccctgagagcagttgcgacgttaagctcgttgagaaatcctttgagacggatacaaatcttaatttccagaatcttagtgtaataggtttccgaatcctgctgcttaaagtagccggtttcaacttgctgatgactttgagactttggtccagc

SEQ ID NO: 33 - DNA, 954 bp, Artificial Sequence
  beta chain
atgacgataaggcttctgtgctatatggggttttatttccttggtgcaggcttgatggaggcagatatctaccaaactccacgatatctggtcattggaaccgggaagaagattacattggaatgctcccagacgatgggccatgacaagatgtactggtaccaacaagatccgggaatggagctccaccttattcactactcctacggggtgaactccacggagaagggggacctgtcttccgaaagtacagtcagccgaatcaggaccgaacatttccccctgactctggagtcagcgcgaccgtctcatacaagccaatatctttgcgcctcatctgaagcgcgcggattggctgagttcacagacacacaatatttcggcccaggtacaagactcacagttttggaggacctgaagaacgtgttcccgccggaagtggcagtcttcgagcctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 34 - DNA, 813 bp, Artificial Sequence
  alpha chain
atgaagaccttcgccggcttcagcttcctgttcctgtggctgcagctggactgtatgagcagaggcgaggatgtggaacagagcctgtttctgagcgtcagagaaggcgacagcagcgtgatcaactgcacctacaccgacagctccagcacctacctgtactggtacaagcaagaacctggcgccggactgcagctgctgacctacatcttcagcaacatggacatgaagcaggaccagcggctgaccgtgctgctgaacaagaaggataagcacctgagcctgcggatcgccgatacacagacaggcgactccgccatctacttttgcgccgagatcaagcggacagccctgatctttggcaagggcacaacactgagcgtgtccagcaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagacagtgctggacatgcggagcatggacttcaagagcaatagcgccgtggcctggtccaacaagagcgattttgcctgcgccaacgccttcaacaacagcatcatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggctttaacctgctgatgaccctgagactgtggtctagc

SEQ ID NO: 35 - DNA, 942 bp, Artificial Sequence
  beta chain
atgggcttcagactgctgtgctgcgtggccttttgtctgcttggagccggacctgtggatagcggagtgacacagacccctaaacacctgatcaccgccacaggccagcgcgtgacactgagatgttctccaagatccggcgacctgtccgtgtattggtatcagcagtctctggaccagggcctgcagttcctgatccagtactacaacggcgaggaaagagccaagggcaacatcctggaaagattcagcgcccagcagttccccgatctgcacagcgaactgaacctgtctagcctggaactgggcgacagcgccctgtatttttgtgcctctggcagcggcatccggcaagagacacagtattttggccctggcacacggctgctggtgctggaagatctgaagaacgtgttcccacctgaggtggccgtgtttgagccttctgaggccgagatctctcacacccagaaagccacactcgtgtgtctggccaccggcttctatcccgatcacgtggaactgtcttggtgggtcaacggcaaagaggtgcacagcggcgttagcacagaccctcagcctctgaaagagcagcccgctctgaacgacagcagatactgtctgtccagcaggctgagagtgtccgccaccttctggcagaaccccagaaaccacttcagatgccaggtccagttctacggcctgagcgagaatgacgagtggacccaggatagggccaagcctgtgactcagattgtgtctgccgaagcctggggcagagccgattgtggctttacaagcgagagctaccagcagggcgtgctgagcgccacaatcctgtatgagatcctgctgggcaaagccactctgtacgctgtgctggtgtctgccctggtgctgatggccatggtcaagagaaaggactccagaggcttaattaat

SEQ ID NO: 36 - DNA, 819 bp, Artificial Sequence
  alpha chain
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgccctctaactttggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 37 - DNA, 948 bp, Artificial Sequence
  beta chain
atgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagccctatatacaggggaagatagcaatcagccccagcattttggtgatgggactcgactctccatcctagaggacctgaagaacgtgttcccgccggaagtggcagtcttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 38 - DNA, 822 bp, Artificial Sequence
  alpha chain
atggcctgccccggcttcctgtgggccctggtgatcagcacctgcctggagttcagcatggcccagaccgtgacccagagccagcccgagatgagcgtgcaggaggccgagaccgtgaccctgagctgcacctacgacaccagcgagagcgactactacctgttctggtacaagcagccccccagcaggcagatgatcctggtgatcaggcaggaggcctacaagcagcagaacgccaccgagaacaggttcagcgtgaacttccagaaggccgccaagagcttcagcctgaagatcagcgacagccagctgggcgacgccgccatgtacttctgcgcctacaggagcgccgtgaacgccaggctgatgttcggcgacggcacccagctggtggtgaagcccaacatccagaaccccgaccccgccgtgtaccagctgagggacagcaagagcagcgacaagagcgtgtgcctgttcaccgacttcgacagccagaccaacgtgagccagagcaaggacagcgacgtgtacatcaccgacaagaccgtgctggacatgaggagcatggacttcaagagcaacagcgccgtggcctggagcaacaagagcgacttcgcctgcgccaacgccttcaacaacagcatcatccccgaggacaccttcttccccagccccgagagcagctgcgacaagctggtggagaagagcttcgagaccgacaccaacctgaacttccagaacctgagcgtgatcggcttcagaatcctgctgctgaaggtggccggcttcaacctgctgatgaccctgaggctgtggagcagc

SEQ ID NO: 39 - DNA, 942 bp, Artificial Sequence
  beta chain
atgaccatcaggctgctgtgctacatgggcttctacttcctgggcgccggcctgatggaggccgacatctaccagacccccaggtacctggtgatcggcaccggcaagaagatcaccctggagtgcagccagaccatgggccacgacaagatgtactggtaccagcaggaccccggcatggagctgcacctgatccactacagctacggcgtgaacagcaccgagaagggcgacctgagcagcgagagcaccgtgagcaggatcaggaccgagcacttccccctgaccctggagagcgccaggcccagccacaccagccagtacctgtgcgccagcaggggcctggccgagttcaccgacacccagtacttcggccccggcaccaggctgaccgtgctggacctgaagaacgtgttcccgccggaagtggcagtcttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 40 - DNA, 825 bp, Artificial Sequence
  alpha chain
atggcctgccccggcttcctgtgggccctggtgatcagcacctgcctggagttcagcatggcccagaccgtgacccagagccagcccgagatgagcgtgcaggaggccgagaccgtgaccctgagctgcacctacgacaccagcgagagcgactactacctgttctggtacaagcagccccccagcaggcagatgatcctggtgatcaggcaggaggcctacaagcagcagaacgccaccgagaacaggttcagcgtgaacttccagaaggccgccaagagcttcagcctgaagatcagcgacagccagctgggcgacgccgccatgtacttctgcgcctacaggagcgccctgaacgccaggctgatgttcggcgacggcacccagctggtggtgaagcccaacatccagaaccccgaccccgccgtgtaccagctgagggacagcaagagcagcgacaagagcgtgtgcctgttcaccgacttcgacagccagaccaacgtgagccagagcaaggacagcgacgtgtacatcaccgacaagaccgtgctggacatgaggagcatggacttcaagagcaacagcgccgtggcctggagcaacaagagcgacttcgcctgcgccaacgccttcaacaacagcatcatccccgaggacaccttcttccccagccccgagagcagctgcgacgtgaagctggtggagaagagcttcgagaccgacaccaacctgaacttccagaacctgagcgtgatcggcttcagaatcctgctgctgaaggtggccggcttcaacctgctgatgaccctgaggctgtggagcagc

SEQ ID NO: 41 - DNA, 954 bp, Artificial Sequence
  beta chain
atgaccatcaggctgctgtgctacatgggcttctacttcctgggcgccggcctgatggaggccgacatctaccagacccccaggtacctggtgatcggcaccggcaagaagatcaccctggagtgcagccagaccatgggccacgacaagatgtactggtaccagcaggaccccggcatggagctgcacctgatccactacagctacggcgtgaacagcaccgagaagggcgacctgagcagcgagagcaccgtgagcaggatcaggaccgagcacttccccctgaccctggagagcgccaggcccagccacaccagccagtacctgtgcgccagcagcgaggccaggggcctggccgagttcaccgacacccagtacttcggccccggcaccaggctgaccgtgctggaggacctgaagaacgtgttcccgccggaagtggcagtcttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 42 - DNA, 840 bp, Artificial Sequence
  alpha chain
atggccatgctgctgggcgccagcgtgctgatcctgtggctgcagcccgactgggtgaacagccagcagaagaacgacgaccagcaggtgaagcagaacagccccagcctgagcgtgcaggagggcaggatcagcatcctgaactgcgactacaccaacagcatgttcgactacttcctgtggtacaagaagtaccccgccgagggccccaccttcctgatcagcatcagcagcatcaaggacaagaacgaggacggcaggttcaccgtgttcctgaacaagagcgccaagcacctgagcctgcacatcgtgcccagccagcccggcgacagcgccgtgtacttctgcgccgcccagatctacaaccagggcggcaagctgatcttcggccagggcaccgagctgagcgtgaagcccaacatccagaaccccgaccccgccgtgtaccagctgagggacagcaagagcagcgacaagagcgtgtgcctgttcaccgacttcgacagccagaccaacgtgagccagagcaaggacagcgacgtgtacatcaccgacaagaccgtgctggacatgaggagcatggacttcaagagcaacagcgccgtggcctggagcaacaagagcgacttcgcctgcgccaacgccttcaacaacagcatcatccccgaggacaccttcttccccagccccgagagcagctgcgacgtgaagctggtggagaagagcttcgagaccgacaccaacctgaacttccagaacctgagcgtgatcggcttcagaatcctgctgctgaaggtggccggcttcaacctgctgatgaccctgaggctgtggagcagc

SEQ ID NO: 43 - DNA, 936 bp, Artificial Sequence
  beta chain
atgggcatcaggctgctgtgcagggtggccttctgcttcctggccgtgggcctggtggacgtgaaggtgacccagagcagcaggtacctggtgaagaggaccggcgagaaggtgttcctggagtgcgtgcaggacatggaccacgagaacatgttctggtacaggcaggaccccggcctgggcctgaggctgatctacttcagctacgacgtgaagatgaaggagaagggcgacatccccgagggctacagcgtgagcagggagaagaaggagaggttcagcctgatcctggagagcgccagcaccaaccagaccagcatgtacctgtgcgccagcagcttcagcagcggcaagcagtacttcggccccggcaccaggctgaccgtgaccgaggacctgaagaacgtgttcccccccgaggtggccgtgttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 44 - DNA, 813 bp, Artificial Sequence
  alpha chain
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagatcaaaagaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 45 - DNA, 942 bp, Artificial Sequence
  beta chain
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcggctcaggggagcaagagacccagtacttcgggccaggcacgcggctcctggtgctcgaggacctgaaaaacgtgttcccacccgaggtcgctgtgttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 46 - DNA, 813 bp, Artificial Sequence; identical to SEQ ID NO: 44
  alpha chain

SEQ ID NO: 47 - DNA, 951 bp, Artificial Sequence
  beta chain
atgagcctcgggctcctgtgctgtggggccttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttccggctctagcggggtgtaccaagagacccagtacttcgggccaggcacgcggctcctggtgctcgaggacctgaaaaacgtgttcccacccgaggtcgctgtgttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 48 - DNA, 813 bp, Artificial Sequence
  alpha chain
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagaactggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 49 - DNA, 942 bp, Artificial Sequence
  beta chain
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcgggtcagggattcggcaagagacccagtacttcgggccaggcacgcggctcctggtgctcgaggacctgaaaaacgtgttcccacccgaggtcgctgtgttcgaacctagcgaggcagaaataagccatactcagaaagcgaccctggtatgcctggccacaggattctatcccgaccatgtcgagctcagctggtgggtaaacggtaaagaggtacatagtggtgtgtcaacagaccctcagccccttaaagagcaacctgcccttaacgatagtcggtattgcttgtcatctaggctcagagtttccgccacgttttggcagaatcctaggaaccattttcgctgccaggtccaattctacgggctctcagaaaacgacgaatggacccaggacagagcgaagccagtaactcagatagtcagcgctgaagcatggggacgagcggattgcggatttacgagcgagtcctaccagcaaggcgttctctccgccaccatcctttacgagattttgctcgggaaggccacattgtacgccgtattggtaagtgcgcttgtcctgatggccatggtcaagagaaaagattctagaggcttaattaat

SEQ ID NO: 50 - DNA, 66 bp, Artificial Sequence
  P2A
ggaagcggcgctacgaactttagtctgctcaaacaggctggggatgtagaggaaaatccggggcct

SEQ ID NO: 51 - DNA, 63 bp, Artificial Sequence
  T2A
ggctccggcgagggcaggggaagtcttctaacatgcggggacgtggaggaaaatcccggccca

SEQ ID NO: 52 - DNA, 720 bp, Artificial Sequence
  eGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 53 - protein, 22 aa, Artificial Sequence
  P2A
GSGATNFSLLKQAGDVEENPGP

SEQ ID NO: 54 - protein, 21 aa, Artificial Sequence
  T2A
GSGEGRGSLLTCGDVEENPGP

SEQ ID NO: 55 - protein, 239 aa, Artificial Sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK